SEQUENCE LISTING (18 sequences)

SEQ ID NO: 1 - DNA, 291 bp, Homo sapiens
ggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagc

SEQ ID NO: 2 - DNA, 24 bp, Homo sapiens
aactctgaagtcatcctgccagtc

SEQ ID NO: 3 - DNA, 23 bp, Artificial
  Artificial sequence probe
atggcggttctcatgctggcaac

SEQ ID NO: 4 - DNA, 24 bp, Homo sapiens
cttcaggttgtggtaaacctctgc

SEQ ID NO: 5 - DNA, 23 bp, Artificial
  synthetic oligonucleotide
  misc_feature (23)..(23): 2′-TIPS blocking group
  misc_feature (23)..(23): 2′-TIPS modification
atggcggttctcatgctggcaac

SEQ ID NO: 6 - DNA, 23 bp, Artificial
  synthetic oligonucleotide
  misc_feature (23)..(23): 2′-TBDPS blocking group
  misc_feature (23)..(23): 2′-TBDPS modification
atggcggttctcatgctggcaac

SEQ ID NO: 7 - DNA, 23 bp, Artificial
  synthetic oligonucleotide
  misc_feature (23)..(23): 2′-TIPS blocking group
  misc_feature (23)..(23): 2′-TIPS modification
atggcggttctcatgctggcatc

SEQ ID NO: 8 - DNA, 23 bp, Artificial
  synthetic oligonucleotide
  misc_feature: 2′-BDPS blocking group
  misc_feature: 2′-BDPS modification
atggcggttctcatgctggcatc

SEQ ID NO: 9 - DNA, 23 bp, artificial
  synthetic oligonucleotide
  misc_feature (22)..(22): dU residue with an aminooxy quencher modification
  misc_feature (23)..(23): 2′-TIPS modification
atggcggttctcatgctggcauc

SEQ ID NO: 10 - DNA, 23 bp, Artificial
  synthetic oligonucleotide
  misc_feature (22)..(22): dU residue with an aminooxy quencher
  misc_feature (23)..(23): 2′-TBDPS modification
atggcggttctcatgctggcauc

SEQ ID NO: 11 - DNA, 23 bp, Artificial
  synthetic oligonucleotide
  misc_feature (22)..(22): dU residue with an aminooxy quencher modification
  misc_feature (23)..(23): methyl dC
atggcggttctcatgctggcauc

SEQ ID NO: 12 - DNA, 23 bp, Artificial
  synthetic oligonucleotide
  misc_feature (22)..(22): dU residue with an aminooxy quencher
atggcggttctcatgctggcauc

SEQ ID NO: 13 - DNA, 23 bp, Artificial
  synthetic oligonucleotide
atggcggttctcatgctggcatc

SEQ ID NO: 14 - DNA, 23 bp, Artificial
  synthetic oligonucleotide
  misc_feature (23)..(23): methyl C
atggcggttctcatgctggcaac

SEQ ID NO: 15 - DNA, 23 bp, Artificial
  synthetic oligonucleotide
  misc_feature (23)..(23): methyl C
atggcggttctcatgctggcatc

SEQ ID NO: 16 - DNA, 23 bp, Artificial
  synthetic oligonucleotide
  misc_feature (1)..(1): 5′ FAM modification
  misc_feature (23)..(23): aminooxy quencher modification
atggcggttctcatgctggcaac

SEQ ID NO: 17 - DNA, 23 bp, Artificial
  synthetic oligonucleotide
  misc_feature (1)..(1): 5′ FAM modification
  misc_feature (9)..(9): dU residue with aminooxy quencher modification
atggcggtuctcatgctggcaac

SEQ ID NO: 18 - DNA, 26 bp, Artificial
  synthetic oligonucleotide
  misc_feature (1)..(1): 5′ FAM modification
  misc_feature (23)..(23): 3′ quencher modification
aatggcggttctcatgctggcaacaa

(19) United States
(12) Patent Application Publication
(10) Pub. No.: US 2007/0218490 A1
(43) Pub. Date: Sep. 20, 2007

(54) NUCLEIC ACID MONOMERS WITH 2'-CHEMICAL MOIETIES

(75) Inventors: Andrei Laikhter, Iowa City, IA (US); Joseph A. Walder, Chicago, IL (US); Mark A. Behlke, Coralville, IA (US)

Correspondence Address: JOHN PETRAVICH;INTEGRATED DNA TECHNOLOGIES, INC., 8180 MCCORMICK BLVD., SKOKIE, IL, 60076-2920, US

(73) Assignee: INTEGRATED DNA TECHNOLOGIES, INC., Skokie, IL (US)

(21) Appl. No.: 11/686,894

(22) Filed: Mar. 15, 2007

Related U.S. Application Data
(60) Provisional application No. 60/782,582, filed on Mar. 15, 2006.

(51) Int. Cl. C12Q 1/68; C12P 19/34; C07H 21/04

(52) U.S. Cl. 435/6; 435/91.2; 536/25.32; 534/727

(57) ABSTRACT

The invention provides nucleic acid monomers with a 2′-modification that are useful for the incorporation of dyes or blocking groups. The monomers can be incorporated on the 3′-end of a dual labeled probe to inhibit PCR polymerase extension during PCR. The polymerase is inhibited from extending the probe at the 3′-hydroxyl group when the monomer is present; there is no need to add a chemical moiety to the 3′-hydroxyl or remove the 3′-hydroxyl. The monomers can also be incorporated internally or at the 5′-end of the oligonucleotide. A detectable label, such as a fluorescent or quenching dye, can be incorporated on the 2′-position of such monomers.

### FIELD OF THE INVENTION

This invention pertains to compositions and methods useful in the design of oligonucleotides that can be used in DNA probe assays and that are especially useful in monitoring the kinetics of amplification reactions.

### BACKGROUND OF THE INVENTION

Amplification assays are widely used research tools in microbiology to study genetic material. Amplifying DNA sequences is useful in cloning, sequencing, mapping and analyzing gene expression. Polymerase Chain Reaction (PCR) is the most widely used amplification assay. An initial amount of cDNA or DNA is provided by the technician, and the PCR process will produce copies of the desired DNA on a logarithmic scale. Typically in PCR, two oligonucleotide primers that hybridize to opposite strands and flank the region of interest in target DNA are extended using DNA polymerase to produce additional copies of the region of interest. This process is repeated for 30-40 cycles to achieve an exponential amount of the targeted sample.

Real-time PCR is a major advancement over traditional PCR for quantitatively determining the amount of DNA in the initial sample. In real-time PCR, the kinetics of the PCR amplification are measured as the amplification takes place. By measuring the earlier phases of the reaction rather than just the endpoint of the reaction, real-time PCR offers advantages such as higher sensitivity, more precision and less sample processing. Real-time PCR also allows a technician to analyze multiple sequence sites within a target sample.

In one method of real-time PCR, dual-labeled probes having a fluorophore and a quencher dye are used to monitor the kinetics of PCR amplification. In one version of this method, the oligonucleotide probes are designed to hybridize to the 3′-end (“downstream”) of an amplification primer so that the 5′-to-3′ exonuclease activity of a polymerase digests the 5′ end of the probe and cleaves off a dye (either the donor fluorophore or the quencher) from that end. The fluorescence intensity of the sample increases and can be monitored as the probe is digested during the course of the amplification. The 3′-hydroxyl group is capped with a protecting group to prevent probe extension during PCR. The protecting group may also serve as a dye group that is used to monitor the reaction.

Another method of real-time PCR uses a label that emits a greater signal when bound to double-stranded DNA. As more double-stranded amplicons are produced, the dye signal increases. This method is limited in its precision because the dye binds to any double stranded DNA and is not specific to a predetermined target.

Another method of real-time PCR is utilizing a probe that contains a segment that is complementary to the target sequence, but the probe forms a hairpin loop. The fluorophore and quencher are covalently linked while in a loop structure, but they are separated as the sequence attaches to the target sequence, thereby giving a detectable signal as the probe's conformation changes. Hairpin probes are difficult to use because the hairpin itself can adversely affect the kinetics of the binding between the probe and the target sample, and they are more difficult to manufacture.

There are a limited amount of alternatives available for measuring the kinetics of PCR amplification even though it is a ubiquitous biological research tool. New methods should also be chemically stable so that they can be incorporated into PCR and related applications without significant degradation or side reactions. Lastly, the most useful compositions should be easily manufactured.

The invention provides nucleic acid monomers that, when incorporated in an oligonucleotide at the 3′ position, inhibit polymerase extension of the probe. The monomers can also be modified to incorporate a dye group at the 5′- or 3′-end of the oligonucleotide or internally for detection purposes without impairing the hybridization of the probe. Moreover, the monomers and oligonucleotides of the present invention are chemically stable and can be easily manufactured and purified. These and other advantages of the invention, as well as additional inventive features, will be apparent from the description of the invention provided herein.

### BRIEF SUMMARY OF THE INVENTION

The invention provides nucleic acid monomers with a 2′ modification that, when incorporated on the 3′-end in an oligonucleotide, inhibit DNA polymerase extension and block primer function. The polymerase is unable to extend the oligonucleotide at the 3′-hydroxyl group, but there is no need to add a chemical moiety to the 3′-hydroxyl or remove the 3′-hydroxyl. The monomers can also incorporate a detectable label at the 3′-end or 5′-end of the oligonucleotide or for internal labeling, such as a fluorescent or quenching dye on the 2′-position of the monomer. The said dye may serve as a polymerase blocking group. Such modifications can actually increase the stability of the duplex. Conventional 3′ capping is accomplished through a 3′-phosphate which can be removed enzymatically. The current invention is irreversible because it is immune from enzymatic cleavage

The monomer contemplated by this invention is represented by Formula 1. In Formula 1, B represents a nucleobase such as adenine, guanine, cytosine, uracil, thymine or any base analogue which pairs like a conventional base in a Watson-Crick manner, or any modification thereof that is known in the art. Y represents any chemical moiety that, when the monomer is used in a probe, is capable of inhibiting the polymerase from extending the probe through the 3′-hydroxyl. The chemical moiety can be, but is not limited to, a dye, a heteroatom-containing alkyl chain, and acetal chain, a phosphate-containing group, or a silyl group. W represents a phosphodiester bond, a hydroxyl group, a protected hydroxyl group, a nucleotide, an oligonucleotide chain, an —SH— group, a protected —SH— group or a phosphorothioate bond. Z represents a hydroxyl group, a solid support, or a linking group such as a phosphoramidite, a succinate monoester, H-phosphonate or phosphate diester.

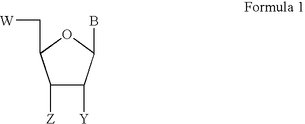

The monomer can also be used at the 5′-end or internally in an oligonucleotide to provide a fluorophore, quencher or any further modification attachment at any position within the oligonucleotide. Internal placement offers the advantage of placing the fluorophore and quencher in closer proximity, thereby enhancing the efficiency of the quenching while the oligonucleotide is intact.

### BRIEF DESCRIPTION OF THE DRAWINGS

FIG. 1 shows serial end-point PCR reaction products produced in control reactions using primer sets For1+For2, For1, or For2 primers with Rev. Products were separated by non-denaturing polyacrylamide gel electrophoresis (PAGE).

FIG. 2 shows serial end-point PCR reaction products produced using primers bearing bulky 2′-modifications. The Rev primer was paired with primers For2-TIPS or For2-TBDPS with or without addition of the For1 primer as competitor. The term “Henol 2′TIPS primer” corresponds to SEQ ID NO: 5, and “HENOL 2′TBDPS” corresponds to SEQ ID NO: 6.

FIG. 3 shows serial end-point PCR reaction products using primers with a small 2′-modification (2′-O-methyl) with or without mismatch present at the (n−1) position or with an internal quencher (dU-aolBFQ). The term “Henol 2′Ome” corresponds to SEQ ID NO: 14, and “Henol-MM-2′Ome” corresponds to SEQ ID NO: 15. The bottom panel of gels corresponds to SEQ ID NO: 11.

FIG. 4 shows real-time qPCR amplification traces assays targeting the human enolase amplicon (SEQ ID NO: 1). Input target amounts were 5×10², 5×10⁴, and 5×10⁶ copies of plasmid DNA. Primers For1 and Rev were employed. Fluorescence quenched probes employed were either standard 3′-end block or the new 2′-block compounds of the invention. All data points were performed in triplicate. Curves were nearly identical and are superimposed. The term “Standard HEnol probe” corresponds to SEQ ID NO: 18, and “HEnol TIPS probe” corresponds to SEQ ID NO: 17.

### DETAILED DESCRIPTION OF THE INVENTION

The invention provides nucleic acid monomers with a 2′ group that, when incorporated in a dual labeled oligonucleotide between about 10 to about 75 monomers long, inhibit polymerase extension of the oligonucleotide and blocks primer function. The 2′-group sterically hinders the polymerase, making the polymerase unable to extend the probe at the 3′-hydroxyl group. The monomers can also incorporate a detectable label, such as a fluorescent or quenching dye on the 2′-position of the sugar ring.

One embodiment of the invention is illustrated in Formula 1.

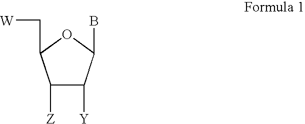

In Formula 1, B represents a nucleobase such as adenine, guanine, cytosine, uracil, thymine or any base analogue which pairs like a conventional base in a Watson-Crick manner, or any modification thereof that is known in the art. Y represents any chemical moiety that, when the monomer is used in a probe, is capable of inhibiting the polymerase from extending the probe through the 3′-hydroxyl. However, if the monomer is used internally, Y does not need to be designed to inhibit the extension of the probe. The chemical moiety can be, but is not limited to, a dye (including a fluorescence quencher), a heteroatom-containing alkyl chain, an acetal chain, a phosphate-containing group, or a silyl group. W represents a phosphodiester bond, a hydroxyl group, a protected hydroxyl group, a nucleotide, an oligonucleotide chain, an —SH— group, a protected —SH— group or a phosphorothioate bond. Z represents a hydroxyl group, a solid support, or a linking group such as a phosphoramidite, a succinate monoester, H-phosphonate or phosphate diester.

When Y is a chemical moiety that functions as a dye, the dye and the linking group attaching the dye to the monomer optionally inhibiting extension of the 3′-end by a polymerase (not required when the monomer is internally placed or placed on the 5′-end). Alternatively, any dyes necessary for the operation of the probe can be located elsewhere, and Y can be a chemical moiety that optionally functions simply as a blocking group to inhibit polymerase extension. A fluorescent dye (fluorophore) or a fluorescent-quenching dye (quencher) can therefore be referred to as a fluorophore or quencher, and they are also subclasses of what can be considered a blocking group. Alternative reporter groups are also contemplated with the present invention. In addition to fluorophores, such reporter groups could be a radiolabel, a hapten, or other reporter groups well known in the art. Suitable blocking groups include an alkyl chain with a substituted heteroatom, an acetal chain, a phosphate-containing group, or a silyl group. Suitable silyl groups include triisopropyl silyl (TIPS), tert-butyldimethylsilyl (TBDMS), or tert-butyldiphenylsilyl (TBDPS).

Multiple monomers as depicted in Formula 1 could be present in an oligonucleotide, and a monomer of the present invention can be located at any position of a given oligonucleotide.

In another embodiment, the invention is represented by Formula 2.

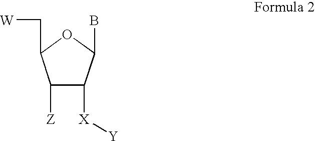

In Formula 2, B represents a nucleobase such as adenine, guanine, cytosine, uracil, thymine, any base analogue which pairs like a conventional base in a Watson-Crick manner, or any modification thereof that is known in the art. X represents a heteroatom such as an oxygen or sulfur, an alkyl group or an amine group. The bond between X and the 2′-carbon of the ribose ring can be a single or a double bond. Y represents any chemical moiety that, when the monomer is used in a probe, is capable of inhibiting the polymerase from extending the probe through the 3′-hydroxyl. The chemical moiety can be, but is not limited to, a dye, a heteroatom-containing alkyl chain, an acetal chain, a phosphate-containing group, or a silyl group. W represents a phosphodiester bond, a hydroxyl group, a protected hydroxyl group, a nucleotide, an oligonucleotide chain, an —SH— group, a protected —SH— group or a phosphorothioate bond. Z represents a hydroxyl group, a solid support, or a linking group such as a phosphoramidite, a succinate monoester, H-phosphonate or phosphate diester.

In another embodiment, the 2′-blocking group is a quencher containing a novel nucleophile group, such as an aminooxy group. See Formula 3. Such a group would allow the dye to react and become covalently attached to electrophilic groups, such as ketone groups. See Laikhter et al., U.S. patent application Ser. No. 11/438,606. An aminooxy link offers increased stability during thermocyclic conditions because the reaction occurs rapidly under mild conditions to offer an extremely stable linkage. The monomer would have a 2′ ketone attachment group, and this monomer could be used generally in any position on an oligonucleotide to provide a means for attaching a modification.

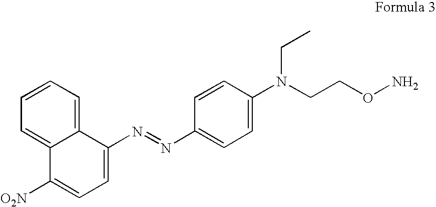

The reagents in Formulas 1 and 2 can be used to derivatize a solid support, and the derivatized support can serve as the foundation for oligonucleotide synthesis by standard methods. A linking group, Z, such as phosphoramidite, an H-phosphonate or phosphate diester, can also be used to introduce a label into an internal position of the oligonucleotide. The method is generally applicable to the attachment of the quencher to any solid support typically used in oligonucleotide synthesis (but not essentially), including but not limited to polystyrene and polypropylene and controlled pore glass. The solid support-bound monomer and trityl-protected, phosphoramidite dye can both be used conveniently in conjunction with automated oligonucleotide synthesizers to directly incorporate the dye into oligonucleotides during their chemical synthesis. Disclosed monomers can be used for post-synthetic modification of oligonucleotides. Such precursors and the oligonucleotides prepared with them are also contemplated by the present invention.

For purposes of this invention the term “linking group” refers to a chemical group that is capable of reacting with a “complementary functionality” of a reagent. When the complementary functionality is an amine, preferred linking groups include such groups as isothiocyanate, sulfonylchloride, 4,6-dichlorotriazinyl, carboxylate, succinimidyl ester, other active carboxylate, e.g., —C(O)halogen, —C(O)OC₁₋₄ alkyl, or —C(O)OC(O)C₁₋₄ alkyl, amine, lower alkylcarboxy or —(CH₂)ₘN⁺(CH₃)₂(CH₂)ₘCOOH, wherein m is an integer ranging from 2 to 12. When the complementary functionality is a 5′-hydroxyl group of an oligonucleotide, the preferred linking group is a protected phosphoramidite. When the complementary functionality is sulfhydryl, the linking group can be a maleimide, halo acetyl, or iodoacetamide for example. See R. Haugland (1992) Molecular Probes Handbook of Fluorescent Probes and Research Chemicals, Molecular Probes, Inc., disclosing numerous modes for conjugating a variety of dyes to a variety of compounds which sections are incorporated herein by reference.

The invention also is directed to nucleic acid compositions containing dye pairs. Suitable dye pairs include a quencher composition and a fluorophore known and disclosed in the literature. Suitable fluorescent dyes in the dye pair are those that emit fluorescence that can be quenched by the quencher of the dye pair. In certain embodiments, the dye pair can be attached to a single compound, such as an oligonucleotide. In other embodiments, the fluorescent reporter dye and the quencher can be on different molecules. The monomers of the invention can be on the 3′-end, the 5′-end or internal within the oligonucleotide and therefore can provide an internally labeled probe that can optimize the distance of the dye pairs for optimal signal.

Many suitable forms of these fluorescent reporter dyes are available and can be used depending on the circumstances. With xanthene compounds, substituents can be attached to xanthene rings for bonding with various reagents, such as for bonding to oligonucleotides. For fluorescein and rhodamine dyes, appropriate linking methodologies for attachment to oligonucleotides have also been described. See for example, Khanna et al. U.S. Pat. No. 4,439,356; Marshall (1975) Histochemical J., 7:299-303; Menchen et al., U.S. Pat. No. 5,188,934; Menchen et al., European Patent Application No. 87310256.0; and Bergot et al., International Application PCT/U590/05565).

Probes having a high signal to noise ratio are desirable for the development of highly sensitive assays. To measure signal to noise ratios of dual-labeled probes, relative fluorescence is measured in a configuration where the quencher and fluorophore are in proximity, e.g. within the Forster distance, and the fluorophore is maximally quenched (background fluorescence or “noise”) and compared with the fluorescence measured when fluorophore and quencher are separated in the absence of quenching (“signal”). The signal to noise ratio of a dye pair of the invention will generally be at least about 2:1 but generally is higher. Signal to noise ratios of about 5:1, 10:1, 20:1, 40:1 and 50:1 are preferred. Ratios of 60:1, 70:1 and even greater than 100:1 can also be obtained. Intermediate signal to noise ratios are also contemplated.

Suitable dye-pairs can be used in many configurations. For example, the dye pair can be placed on nucleic acid oligomers and polymers. In this format, a dye-pair can be placed on an oligomer having a hairpin structure such that the fluorophore and quencher are within the Förster distance and FRET occurs.

In other embodiments, dye pairs can be placed on an oligomer that can adopt a random coil conformation, such that fluorescence is quenched until the oligonucleotide adopts an extended conformation, as when it becomes part of a duplex nucleic acid polymer. In general, the individual dye moieties can be placed at any position of the nucleic acid depending upon the requirements of use.

Nucleic acid oligomers and polymers that include the dye pairs of the invention can be used to detect target nucleic acids. In one method, the individual components of a dye-pair can be on opposing, annealable, self-complementary segments, forming a hairpin, of a single oligonucleotide such that when the oligonucleotide anneals to itself in the absence of target sequences, FRET or static quenching occurs. The oligonucleotide is constructed in such a way that the internal annealing is disrupted and fluorescence can be observed when it hybridizes to nucleic acid polymers having sufficient complementarity. Such an oligonucleotide can be used to rapidly detect nucleic acid polymers having sequences that bind to the oligonucleotide.

Oligonucleotide probes lacking self-complementarity can also be utilized in a similar manner. For example, a quencher and fluorophore can be placed on an oligonucleotide that lacks the self-annealing property such that the random-coil conformation of the oligonucleotide keeps the fluorophore and quencher within a suitable distance for fluorescence quenching. Such oligonucleotides can be designed so that when they anneal to desired target nucleic acid polymers the fluorophore and quencher are more separated and the spectral characteristics of the fluorophore become more apparent.

Other DNA binding formats are also possible. For example, two oligonucleotides can be designed such that they can anneal adjacent to each other on a contiguous length of a nucleic acid polymer. The two probes can be designed such that when they are annealed to such a nucleic acid polymer a quencher on one of the oligonucleotides is within a sufficient proximity to a fluorophore on the other oligonucleotide for FRET to occur. Binding of the oligonucleotides to the nucleic acid polymer can be followed as a decrease in the fluorescence of the fluorophore. In another embodiment, the quencher need not be a dark quencher but rather itself could emit fluorescence at a longer wavelength.

In another embodiment, a set of oligonucleotides that anneal to each other can be configured such that a quencher and a fluorophore are positioned within the Förster distance on opposing oligonucleotides. Incubation of such an oligonucleotide duplex with a nucleic acid polymer that competes for binding of one or both of the oligonucleotides would cause a net separation of the oligonucleotide duplex leading to an increase in the fluorescent signal of the reporter dye. To favor binding to the polymer strands, one of the oligonucleotides could be longer or mismatches could be incorporated within the oligonucleotide duplex.

The oligonucleotides of this invention can also be used in a polynomial amplification assay format (see Behlke, et al., U.S. Pat. No. 7,112,406), or in assays wherein the primers serve the function of both template and amplification (see Behlke, et al., U.S. patent application Ser. No. 11/563,072).

These assay formats can easily be extended to multi-reporter systems that have mixtures of oligonucleotides in which each oligonucleotide has a fluorophore with a distinct spectrally resolvable emission spectrum. The binding of individual oligonucleotides can then be detected by determining the fluorescent wavelengths that are emitted from a sample. Such multi-reporter systems can be used to analyze multiple hybridization events in a single assay.

Oligonucleotides can also be configured with the disclosed monomers such that they can be used to monitor the progress of PCR reactions without manipulating the PCR reaction mixture (i.e., in a closed tube format). One such assay utilizes an oligonucleotide that is labeled with a fluorophore and a quencher in a configuration such that fluorescence is substantially quenched. The oligonucleotide is designed to have sufficient complementarity to a region of the amplified nucleic acid so that it will specifically hybridize to the amplified product. The hybridized oligonucleotide is degraded by the 5′-exonuclease activity of Taq polymerase in the subsequent round of DNA synthesis. The oligonucleotide is designed such that as the oligomer is degraded, the members of the dye-pair are separated and fluorescence from the fluorophore can be observed. An increase in fluorescence intensity of the sample indicates the accumulation of amplified product.

Ribonucleic acid polymers can also be configured with fluorophores and quenchers and used to detect single-stranded or double-stranded ribonucleases. For example, a dye-pair can be positioned on opposite sides of an RNase cleavage site in an RNase substrate such that the fluorescence of the fluorophore is quenched (See Walder et al., U.S. Pat. No. 6,773,885). Suitable substrates for detection of single-stranded ribonucleases include nucleic acid molecules that have a single-stranded region that can be cleaved and that have at least one internucleotide linkage immediately 3′ to an adenosine residue, at least one internucleotide linkage immediately 3′ to a cytosine residue, at least one internucleotide linkage immediately 3′ to a guanosine residue and at least one internucleotide linkage next to a uridine residue and optionally can lack a deoxyribonuclease-cleavable internucleotide linkage. Alternatively, any amount between one through the four types of residue can be used, and at any specificity. To conduct the assay, the substrate can be incubated with a test sample for a time sufficient for cleavage of the substrate by a ribonuclease enzyme, if present in the sample. The substrate can be a single-stranded nucleic acid molecule containing at least one ribonucleotide residue at an internal position. Upon cleavage of the internal ribonucleotide residue, the fluorescence of the reporter dye, whose emission was quenched by the quencher, becomes detectable. The appearance of fluorescence indicates that a ribonuclease cleavage event has occurred, and, therefore, the sample contains ribonuclease activity. This test can be adapted to quantitate the level of ribonuclease activity by incubating the substrate with control samples containing known amounts of ribonuclease, measuring the signal that is obtained after a suitable length of time, and comparing the signals with the signal obtained in the test sample.

Generally, any of the described assays could be conducted with positive and negative controls to indicate proper function of the assay.

The invention also provides kits that include in one or more containers, at least one of the disclosed monomer-containing compositions and instructions for its use. Such kits can be useful for practicing the described methods or to provide materials for synthesis of the compositions as described. Additional components can be included in the kit depending on the needs of a particular method. For example, where the kit is directed to measuring the progress of PCR reactions, it can include a DNA polymerase. Where a kit is intended for the practice of the RNase detection assays, RNase-free water could be included. Kits can also contain negative and/or positive controls and buffers.

The following examples further illustrate the invention but, of course, should not be construed as in any way limiting its scope. In particular the following examples demonstrate synthetic methods for obtaining the compounds of the invention. Starting materials useful for preparing the compounds of the invention and intermediates thereof, are commercially available or can be prepared from commercially available materials using known synthetic methods and reagents. All oligonucleotide sequences are written from the 5′-terminus on the left to the 3′-terminus on the right.

### EXAMPLE 1

This example demonstrates the Synthesis of aminooxy activated (1-nitro-4-naphthylazo)-N,ethyl-N-ethanolaniline quencher (3).

The synthesis is as shown in Scheme 1 below. To the solution of 0.36 g (0.1 mmol) alcohol (1) (see U.S. patent application Ser. No. 10/987,608 for synthesis of 1), 0.17 g (0.1 mmol) N-hydroxy-phthalimide, and 0.27 g (0.1 mmol) of triphenylphosphine in 10 mL of THF was added 0.18 mL (0.1 mmol) of DEAD. After overnight stirring the reaction mixture was concentrated under diminished pressure. Flash chromatography with 1:4 EtOAc/hexanes provided 150 mg of (2). TLC: R_{f} 0.75 (EtOAc/hexanes-60/40). ¹H NMR (CDCl₃) δ 9.04 (d, J=8.4 Hz, 1H), 8.68 (d, J=8.4 Hz, 1H), 8.34 (d, J=8.4 Hz, 1H), 8.03 (d, J=8 Hz, 2H), 7.7-7.9 (m, 7H), 6.85 (d, J=8 Hz, 2H), 4.46 (t, J=7.5 Hz, 2H), 3.92 (t, J=7.5 Hz 2H), 3.72 (q, J=8 Hz, 2H), 1.34 (t, J=8 Hz 3H).

The solution of 10 mg (2) in 2 mL of concentrated ammonia solution in ethanol was incubated overnight at 55° C. The solvent was removed under diminished pressure to provide compound (3) that was used further without purification in the synthesis of aminooxy conjugated CPG supports (10) in Example 3.

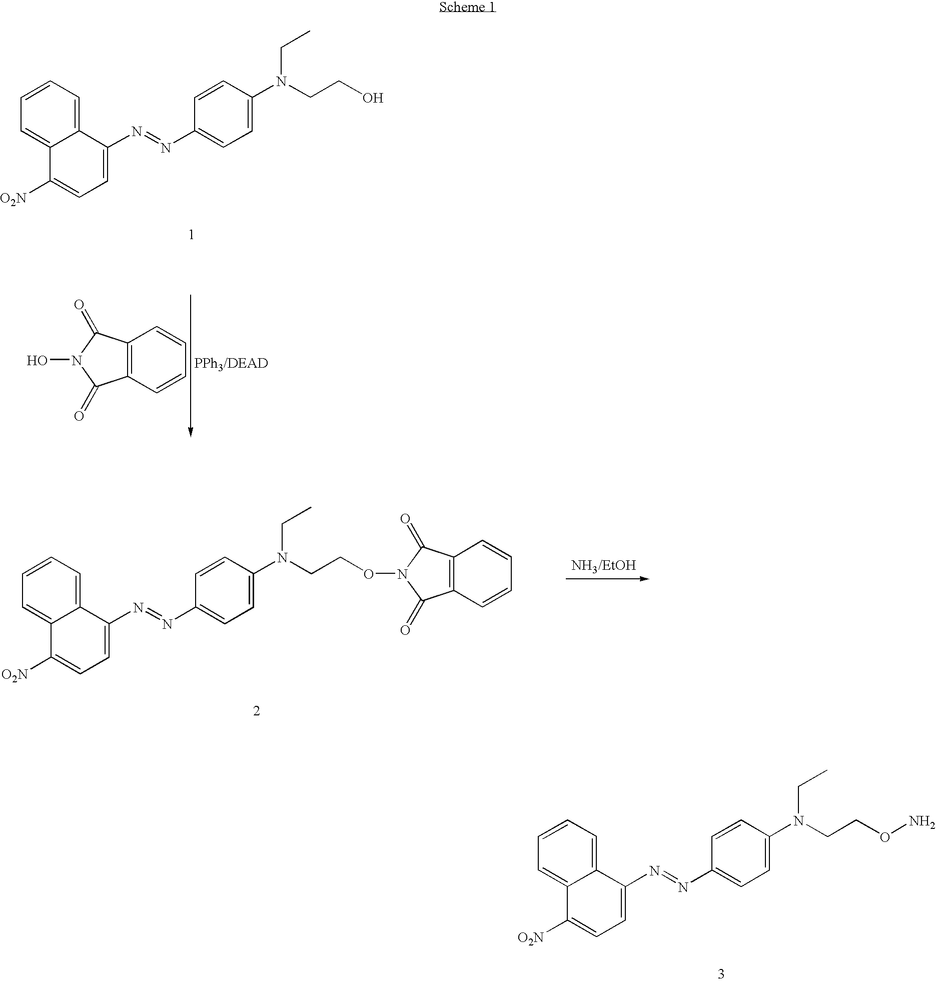

### EXAMPLE 2

This example demonstrates the synthesis of N4-Benzoyl-2′-O-[(3-oxobutyl)methyl]-3′-succinoyl-5′-(4,4′-Dimethoxytrityl)cytidine (8). See Scheme 2.

N⁴-Benzo-1-2′-O-methylthiomethyl-3′,5′-O-(1,1,3,3-tetraisoprolyldisiloxane-1,3-diyl)cytidine (4): To a solution containing 2.88 g (5.91 mmol) of N⁴-Benzoyl-3′,5′-O-(1,1,3,3-tetraisopropyldisiloxane-1,3-diyl)cytidine in 19 mL of DMSO, 19 mL of acetic acid and 12 mL of acetic anhydride were added. After stirring overnight, 50 mL of cold triethylamine (TEA) was added dropwise and the reaction mixture was stirred for 15 mins. 100 mL of water was added and the aqueous layer was extracted with two 100-mL portions of CH₂Cl₂. The organic layers were combined, dried over Na₂SO₄ and then solvent was removed under reduced pressure. The crude product was applied to a silica gel column; elution with a gradient of 3:7-3:2 ethyl acetate-petroleum ether provided N⁴-Benzoyl-2′-O-methylthiomethyl-3′,5′-O-(1,1,3,3-tetraisopropyldisiloxane-1,3-diyl)cytidine (4) as a white foam: yield 2 g (63%); silica gel TLC: R_{f} 0.55 (1:1 ethyl acetate-petroleum ether). ¹H NMR (CDCL₃) δ 8.63 (bs, 1H), 8.36 (d, J=7 Hz, 1H), 7.89 (bs, 2H), 7.50-7.64 (m, 4H), 5.85 (s. 1H), 5.15 (d, J=11 Hz, 1H), 5.01 (d, J=11 Hz, 1H), 4.40 (d, J=2 Hz, 1H), 4.31 (d, J=13 Hz, 1H), 4.22 (d, J=2 Hz, 2H), 4.01 (d, J=13 Hz, 1H), 2.21 (s, 3H), 1.00-1.14 (m, 28H).

N⁴-Benzoyl-2′-O-[(3-oxobutyl)methyl]-3′,5′-O-(1,1,3,3-tetraisopropyldisiloxane-1,3-diyl)cytidine (5): To a stirred solution containing 1.6 g (2.46 mmol) of N⁴-Benzoyl-2′-O-methylthiomethyl-3′,5′-O-(1,1,3,3-tetraisopropyldisiloxane-1,3-diyl)cytidine (4), 1.1 g (12.3 mmol) of 4-hydroxy-2-butanone, 2.38 g (7.38 mmol) of Bu₄NBr and molecular sieves in 30 mL of CH₂Cl₂, 1.65 g (7.38 mmol) of CuBr₂ was added. After stirring for 12 hrs, TLC showed a lower UV spot. 100 mL of aq. 5% NaCO₃ was added and the aqueous layer was extracted with two 100-mL portions of CH₂Cl₂. The organic layers were combined, dried over Na₂SO₄ and removed under reduced pressure. The crude product was applied to a silica gel column; elution with a gradient of 3:6:1-5:4:1 ethyl acetate-petroleum ether-TEA provided N⁴-Benzoyl-2′-O-[(2-butanone-4-hydroxy)methyl]-3′,5′-O-(1,1,3,3-tetraisopropyldisiloxane-1,3-diyl)cytidine (5) as an oil: yield 1.37 g (81%); silica gel TLC: R_{f} 0.45 (8:10:1 ethyl acetate-petroleum ether-TEA). ¹H NMR (CDCL₃) δ 8.64 (bs, 1H), 8.36 (d, J=7 Hz, 1H), 7.89 (d, J=7 Hz, 2H), 7.60-7.64 (m, 1H), 7.50-7.54 (m, 3H), 5.85 (s. 1H), 5.05 (d, J=7 Hz, 1H), 4.97 (d, J=7 Hz, 1H), 4.27-4.33 (m, 2H), 4.21 (d, J=2 Hz, 2H), 3.99-4.06 (m, 2H), 3.90-3.97 (m, 1H), 2.78 (t, J=6 Hz, 2H), 2.19 (s, 3H), 0.97-1.13 (m, 28H).

N⁴-Benzoyl-2′-O-[(3-oxobutyl)methyl]cytidine (6): To a stirred solution containing 1.37 g (1.99 mmol) of N⁴-Benzoyl-2′-O-[(2-butanone-4-hydroxy)methyl]-3′,5′-O-(1,1,3,3-tetraisopropyldisiloxane-1,3-diyl)cytidine (5) and 0.31 mL (5.47 mmol) of AcOH in 10 mL of THF, 4.37 mL (4.37 mmol) of tetrabutyl ammonium fluoride (TBAF) (1M in THF) was added. After stirring for 45 mins, THF was removed under reduced pressure. The crude product was applied to a silica gel column; elution with a gradient of 0:1-1:9 MeOH-ethyl acetate provided N⁴-Benzoyl-2′-O-[(2-butanone-4-hydroxy)methyl]cytidine (6) as a white foam: yield 0.71 g (80%); silica gel TLC: R_{f} 0.55 (1:9 MeOH-ethyl acetate). ¹H NMR (CDCL₃) δ 8.79 (bs, 1H), 8.30 (d, J=7 Hz, 1H), 7.89 (d, J=8 Hz, 2H), 7.50-7.63 (m, 4H), 5.82 (d, J=3 Hz, 1H), 5.02 (d, J=7 Hz, 1H), 4.85 (d, J=7 Hz, 1H), 4.47-4.50 (m, 1H), 4.39 (dd, J=5 Hz, 1H), 4.19-4.22 (m, 1H), 4.01-4.11 (m, 2H), 3.92 (d, J=12 Hz, 1H), 3.62-3.70 (m, 2H), 3.22 (bs, 1H), 2.62-2.83 (m, 2H), 2.18 (s, 3H).

N⁴-Benzoyl-2′-O-[(3-oxobutyl)methyl]-5′-(4,4′-dimethoxytrityl)cytidine (7): To a stirred solution containing 0.71 g (1.59 mmol) of N⁴-Benzoyl-2′-O-[(2-butanone-4-hydroxy)methyl]cytidine (6) in 16 mL of pyridine, 0.81 g (2.38 mmol) of DMTCl was added. After stirring for 4 hrs, Pyridine was removed under reduced pressure. The crude product was applied to a silica gel column; elution with a gradient of 0:1-3:17 acetonitrile-ethyl acetate provided N⁴-Benzoyl-2′-O-[(2-butanone-4-hydroxy)methyl]-5′-(4,4′-dimethoxytrityl)cytidine (7) as a white foam: yield 0.64 g (54%); silica gel TLC: R_{f} 0.55 (ethyl acetate). ¹H NMR (CDCL₃) δ 8.57 (bs, 1H), 7.88 (d, J=7 Hz, 2H), 7.21-7.63 (m, 14H), 6.89 (d, J=9 Hz, 4H), 5.98 (s, 1H), 5.20 (d, J=7 Hz, 1H), 4.90 (d, J=7 Hz, 1H), 4.51-4.55 (m, 1H), 4.07-4.25 (m, 3H), 3.83 (d, J=1 Hz, 6H), 3.67-3.72 (m, 1H), 3.57-3.65 (m, 2H), 3.38 (d, J=7 Hz, 1H), 2.64-2.85 (m, 2H), 2.19 (s, 3H).

N⁴-Benzoyl-2′-O-[(3-oxobutyl)methyl]-3′-succinoyl-5′-(4,4′-dimethoxytrityl)cytidine (8): To a stirred solution containing 0.64 g (0.85 mmol) of N⁴-Benzoyl-2′-O-[(2-butanone-4-hydroxy)methyl]-5′-(4,4′-dimethoxytrityl)cytidine (7) and 0.14 g (1.11 mmol) of DMAP in 9 mL of CH₂Cl₂, 0.11 g (1.11 mmol) of succinic anhydride was added. After stirring for 7 hrs, 30 mL of CH₂Cl₂ was added and washed with three 20-mL portion of 0.5 M aqueous K₂HPO₄. The organic layer was dried over Na₂SO₄ and then the solvent was removed under reduced pressure, providing N⁴-Benzoyl-2′-O-[(2-butanone-4-hydroxy)methyl]-3′-succinoyl-5′-(4,4′-dimethoxytrityl)cytidine (8) as an off-white powder: yield 0.64 g (88%); silica gel TLC: R_{f} 0.45 (3:17 MeOH—CH₂Cl₂). ¹H NMR (CDCL₃) δ 8.49 (J=7 Hz, 1H), 7.94-7.96 (m, 2H), 7.21-7.72 (m, 14H), 6.86 (dd J=9, 1 Hz, 4H), 6.06 (d, J=2 Hz, 1H), 5.37 (dd, J=8, 5 Hz, 1H), 4.97 (d, J=7 Hz, 1H), 4.82 (d, J=7 Hz, 1H), 4.50 (dd, J=5, 2 Hz, 1H), 4.38 (d, J=8 Hz, 1H), 4.18-4.26 (m, 1H), 3.81 (d, J=1 Hz, 6H), 3.74-3.87 (m, 2H), 3.63-3.68 (m, 1H), 2.57-2.79 (m, 6H), 2.15 (s, 3H).

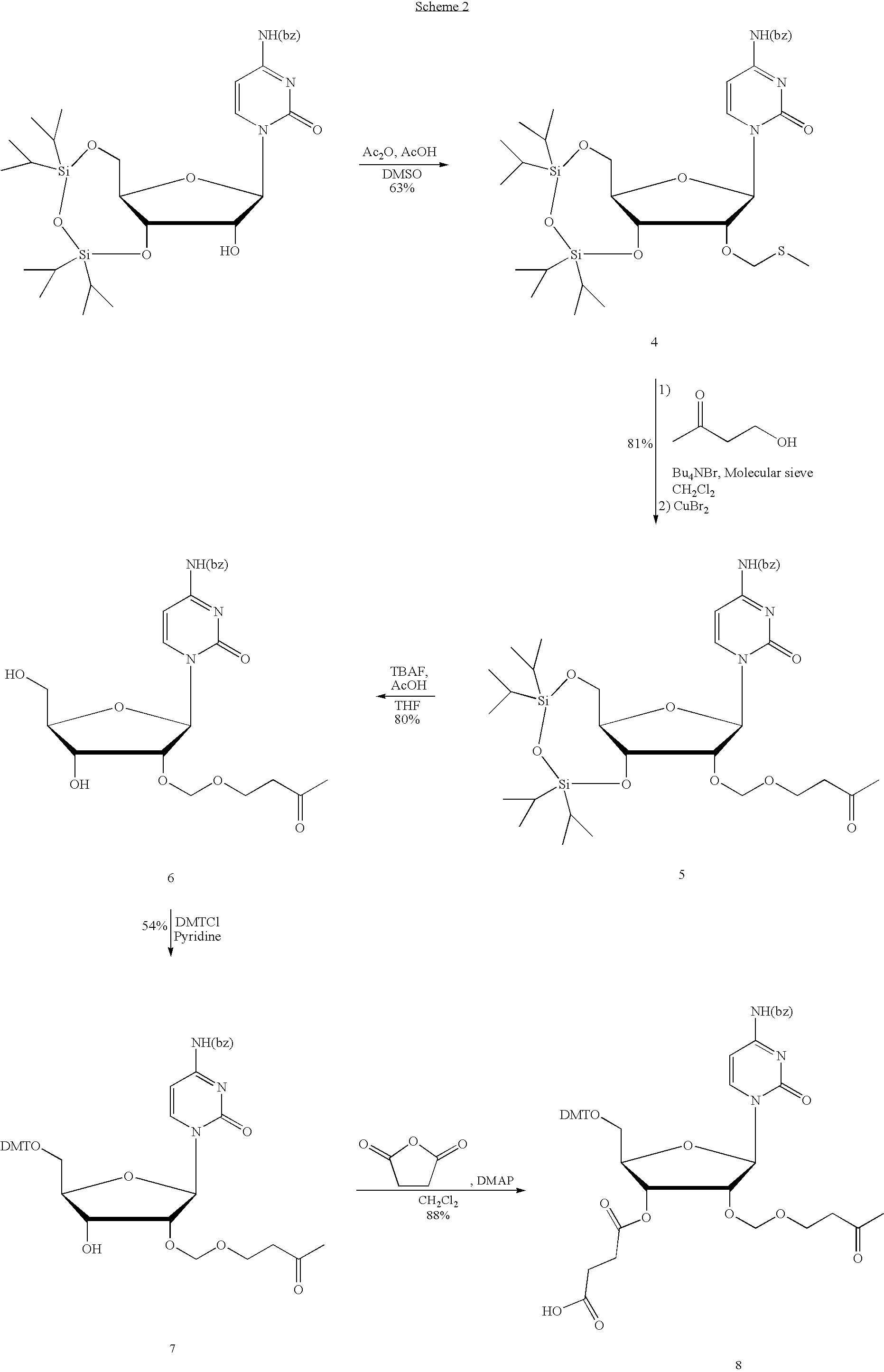

### EXAMPLE 3

This example demonstrates the synthesis of aminooxy conjugated CPG supports with (1-nitro-4-naphthylazo)-N,-ethyl-N-ethanolaniline quencher. The modified solid support (10) can be used for the synthesis of modified oligonucleotides. The synthesis is as shown in Scheme 3 below.

Synthesis of 2′-ketone modified rC CPG supports: To a slurry of long chain amino alkyl (amino-lcaa)-CPG (1.5 g) in 8 mL of acetonitrile were added 0.4 ml of pyridine, 3′-succinyl-2′-ketone-rC nucleoside (8) (0.14 g, 147 μmoles), DIC (0.157 g, 1 mmole), N-hydroxysuccinimide (6 mg, 50 μmoles) and the reaction mixture was placed on rotary shaker. After 12 hrs the resulting CPG (9) was filtered and washed with CH₃CN (5×50 mL). The CPG was then treated with Ac₂O:Melm:Py (10:10:80) (3×30 mL; 5 minutes each treatment). The derivatized CPG (9) washed with CH₃CN (5×30 mL), CH₂Cl₂ (3×30 mL), and dried in vacuum overnight. DMT-loading was usually above 25-30 μmol/g.

Attachment of the chromophore to ketone modified support: To the solution of 10 mg of corresponding aminooxy chromophore (3) in 2 mL of ethanol was added 0.1 g of corresponding ketone modified CPG support (9) and incubated overnight at room temperature. The resulting support (10) was filtered and washed with three 1 ml portions of acetonitrile and then used in oligonucleotide synthesis.

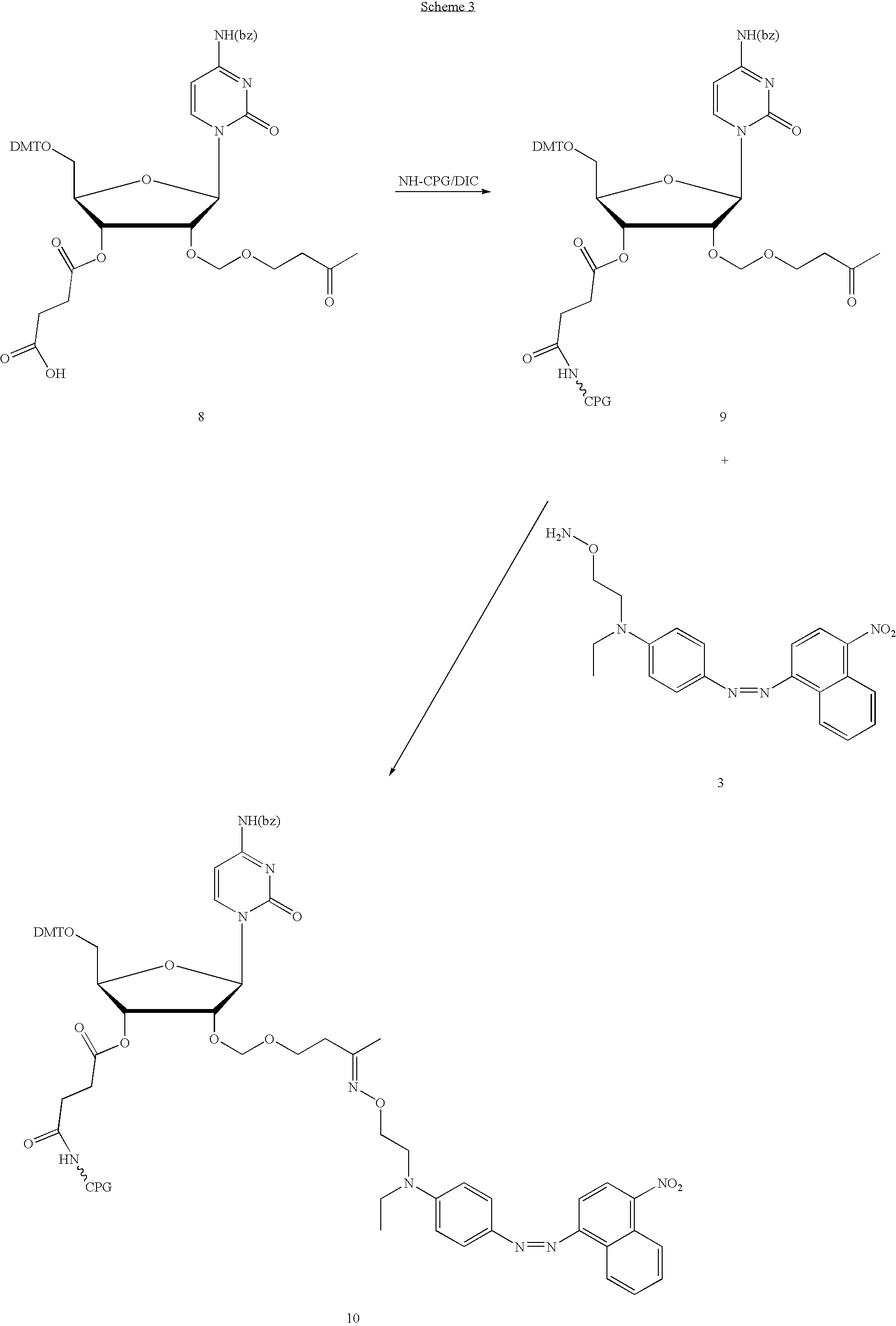

### EXAMPLE 4

This example demonstrates the synthesis of a ketone phosphoramidite (20). The synthesis was performed as shown in Scheme 4 below.

3-Aminopropyl solketal (13): 3-Aminopropyl solketal was synthesized starting from commercially available solketal (11) according to the procedure of Misiura et al. (Misiura, K., Durrant, I., Evans, M. R., Gait, M. J. (1990) Nucleic Acids Research, v. 18, No. 15, pp. 4345-4354.). It was used crude without vacuum distillation for the next step.

N-Fmoc-3-aminopropyl solketal (14): Crude product (13) (12.85 g; 68 mmol) was dissolved in dry CH₃CN (100 mL) with stirring. NaHCO₃ (4.2 g; 50 mmol) was added followed by Fmoc-OSu (16.9 g; 50 mmol). The reaction mixture was stirred at RT overnight. The solvent was evaporated and the oily residue was partitioned between EtOAc (500 mL) and 5% NaHCO₃ (150 mL). The organic layer was separated and washed with 5% NaHCO₃ (2×150 mL), brine (150 mL), and dried over anhydrous Na₂SO₄. The product was isolated by flash chromatography on a silica gel column (5×20 cm) loading from EtOAc:CH₂Cl₂:PE (15:15:70) and eluting with EtOAc:CH₂Cl₂:PE (1:1:2). The isolated product had R_{f} 0.4 by TLC in EtOAc:CH₂Cl₂:PE (1:1:1). Yield: 20.95 g of oil. ¹H NMR (CDCl₃) δ 1.35 (s, 3H), 1.45 (s, 3H), 1.81 (m, 2H), 3.34 (q, 2H), 3.47-3.60 (m, 4H), 3.75 (dd, 1H), 4.07 (dd, 1H), 4.22-4.32 (m, 2H), 4.42 (d, 2H), 5.29 (br.t, 1H), 7.33 (dt, 2H), 7.42 (t, 2H), 7.62 (d, 2H), 7.78 (d, 2H).

1-O—(N-Fmoc-3-aminopropyl)glycerol (15): Crude compound (14) (5 g; 12.1 mmol) was dissolved in THF (15 mL) and treated with 2M HCl (5 mL). The resulting emulsion was shaken at RT with occasional sonication until became homogeneous. It was then left at RT for additional hour. The reaction mixture was concentrated in vacuum, and the resulting oil was co-evaporated with EtOH (3×20 mL). The reaction product (R_{f}˜0.3 in EtOAc:CH₂Cl₂:MeOH (10:10:1)) was isolated by silica gel chromatography (5×20 cm) using a gradient 0-5% MeOH in EtOAc:CH₂Cl₂ (1:1). Fractions containing pure product were pooled and concentrated to give oily residue, which crystallized upon vacuum drying. Yield: 2.64 g of a white solid. ¹H NMR (DMSO-d₆) δ 1.63 (m, 2H), 3.05 (q, 2H), 3.25-3.41 (m, 6H), 3.53-3.60 (m, 1H), 4.21 (t, 1H), 4.30 (d, 2H), 4.47 (t, 1H), 4.60 (d, 1H), 7.27 (t, 1H), 7.33 (dt, 2H), 7.42 (t, 2H), 7.69 (d, 2H), 7.89 (d, 2H).

1-O-DMT-3-O—(N-Fmoc-3-aminopropyl)glycerol (16): 1-O—(N-Fmoc-3-aminopropyl) glycerol ((15), 2.64 g; 7.1 mmol) was dissolved in dry Py (50 mL) and treated with DMT-Cl (2.65 g; 7.8 mmol). The reaction mixture was stirred at RT overnight and quenched with MeOH (5 mL). It was then concentrated to oil under reduced pressure. The residue was dissolved in EtOAc (˜300 mL) and extracted with saturated NaHCO₃ (3×100 mL) followed by brine (100 mL). The organic phase was separated, dried over anhydrous Na₂SO₄ and concentrated to oil. The product was isolated by silica gel chromatography using a gradient 33-66% EtOAc in PE. Yield: 4.03 g (84%) of white foam. TLC showed one spot at R_{f}˜0.6 in EtOAc:PE (2:1). ¹H NMR (CDCl₃) δ 1.68-1.80 (m, 2H), 2.57 (br.d, 1H), 3.17-3.34 (m, 4H), 3.43-3.61 (m, 4H), 3.79 (s, 6H), 3.93-4.00 (m, 1H), 4.22 (t, 1H), 4.41 (d, 2H), 5.20 (br.t, 1H), 6.82-6.86 (m, 4H), 7.21-7.46 (m, 13H), 7.61 (d, 2H), 7.77 (d, 2H).

1-O-DMT-3-O-(3-aminopropyl)glycerol (17): Compound (16) (3.82 g; 5.67 mmol) was dissolved in i-PrOH (100 mL) and sodium borohydride (4 g) was added in portions with stirring. The suspension was heated at 70° C. for 2 hours. TLC analysis in EtOAc:TEA (99:1) revealed the disappearance of the starting material (R_{f}˜0.75) and formation of deprotected product at the start. The reaction was carefully quenched with 10% sodium hydroxide (32 mL), transferred into a separatory funnel and partitioned with 300 mL of ethyl acetate. The organic phase was separated, washed with saturated NaHCO₃ (3×100 mL) followed by brine (100 mL), and dried over sodium sulfate. It was then concentrated in vacuum to give oily residue, which was co-evaporated with dry acetonitrile (50 mL). This crude material was used in the next step without further purification.

Pentafluorophenyl 5-oxohexanoate (18): 5-Oxohexanoic acid (2.6 g; 20 mmol) was dissolved in CH₂Cl₂ (50 mL). N,N-Diisopropylethylamine (10.4 mL, 60 mmol) was added followed by pentafluorophenyl trifluoroacetate (3.61 mL; 21 mmol). The reaction mixture was kept at room temperature for 1 hour and evaporated. The residue was resuspended in EtOAc:Hexanes (1:1) and loaded on a silica gel column (5×20 cm) equilibrated and developed with the same mixture. Fractions containing the product (R_{f} ˜0.7) were pooled and concentrated to give 4.7 g (79%) of yellowish oil after drying in vacuum. ¹H NMR (CDCl₃) δ 2.05 (m, 2H), 2.18 (s, 3H), 2.61 (t, 2H), 2.74 (t, 2H).

1-O-DMT-3-O—(N-(5-oxohexanoyl)-3-aminopropyl)glycerol (19): The crude product (17) was dissolved in dry CH₃CN (50 mL) and treated with N,N-diisopropylethylamine (2.6 mL, 15 mmol) and (18) (1.68 g, 5.67 mmol). The mixture was allowed to react at room temperature for 2 hours. The reaction mixture was evaporated in vacuum and the residue was reconstituted in EtOAc (50 mL). The product was isolated by silica gel chromatography (4×25 cm) loading from 1% TEA in EtOAc and eluting with MeOH:EtOAc:TEA (5:95:1). Fractions containing a single component (R_{f} 0.35) were pooled and concentrated in vacuum to yield the title compound (2.70 g, 85%) as slightly orange oil. ¹H NMR (DMSO-d₆) δ 1.60 (m, 2H), 1.66 (m, 2H), 2.03 (t, 2H), 2.05 (s, 3H), 2.40 (t, 2H), 2.94 (d, 2H), 3.04 (q, 2H), 3.35-3.46 (m, 4H), 3.72-3.79 (m, 7H; OCH₃ singlet at 3.74), 4.84 (d, 1H), 6.88 (d, 4H), 7.19-7.42 (m, 9H), 7.72 (t, 1H).

1-O-DMT-3-O—(N-(5-oxohexanoyl)-3-aminopropyl)glycerol 2-O—(N,N-diisopropyl-(2-cyanoethyl)phosphoramidite) (20): Alcohol (19) (1.35 g, 2.4 mmol) and diisopropylammonium tetrazolide (206 mg, 1.2 mmol) were dissolved in anhydrous CH₃CN (30 mL) under Ar atmosphere. 2-Cyanoethyl N,N,N′,N′-tetraisopropylphosphordiamidite (0.953 mL, 3.0 mmol) was added with stirring at room temperature, and the reaction mixture was stirred overnight. The solvent was evaporated, the residue was reconstituted in EtOAc (200 mL) and washed with saturated NaHCO₃ (3×50 mL) followed with brine (50 mL). The organic layer was dried over anhydrous Na₂SO₄ and the solvent evaporated under reduced pressure. The oily residue was purified by silica gel chromatography eluting with EtOAc:TEA (95:5). Fractions containing pure product, which moves as a double spot on TLC(R_{f} 0.55; EtOAc:TEA (95:5)), were pooled and concentrated in vacuum to give 1.74 g of colorless oil. ¹H NMR (DMSO-d₆) δ 1.01-1.17 (m, 12H), 1.56 (m, 2H), 1.66 (m, 2H), 2.02 (m, 2H), 2.05 (s, 3H), 2.39 (m, 2H), 2.65 (t, 1H), 2.77 (t, 1H), 2.97-3.16 (m, 4H), 3.36-3.81 (m, 15H; OCH₃ singlets at 3.73 and 3.74), 6.88 (m, 4H), 7.19-7.44 (m, 9H), 7.69 (t, 1H). ³¹P NMR (DMSO-d₆) δ 148.19 and 148.64.

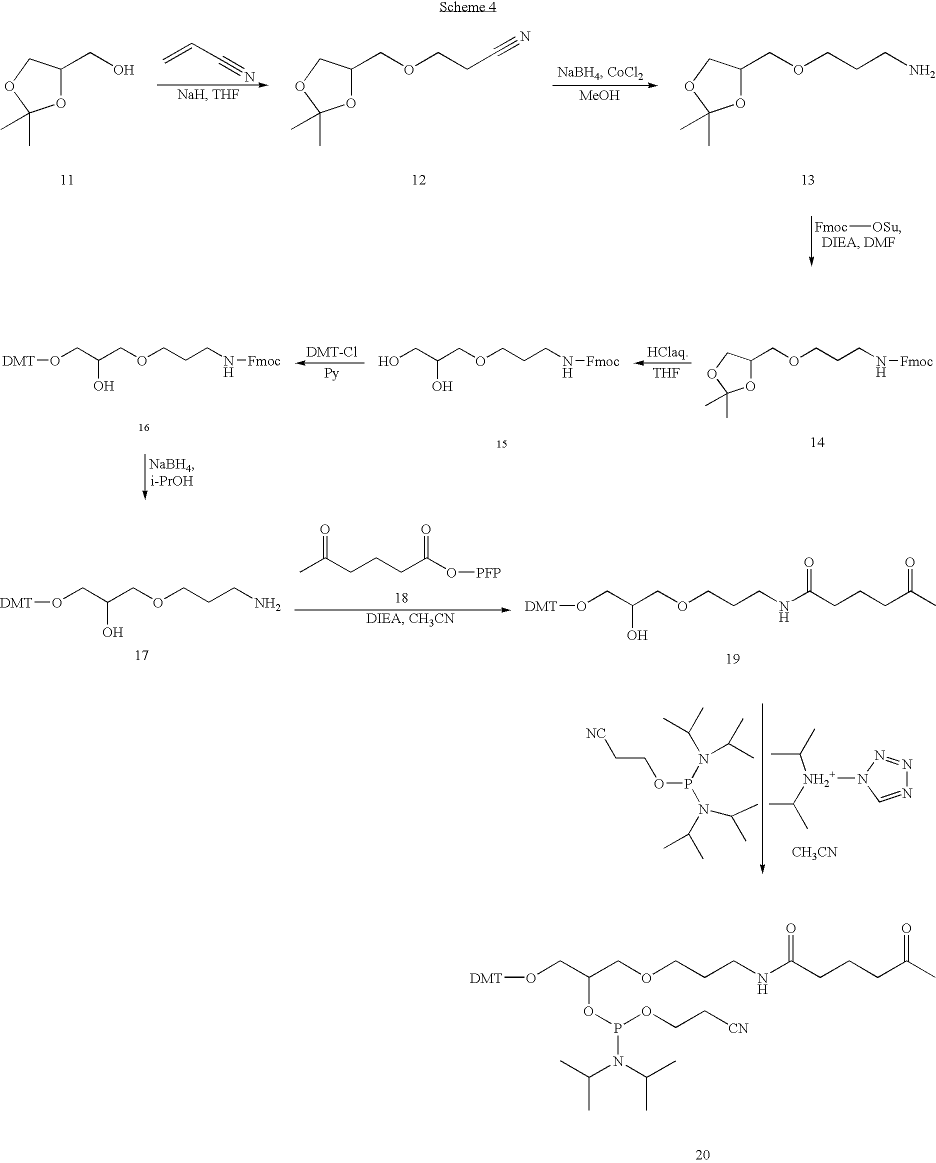

### EXAMPLE 5

This example shows the synthesis of N⁴-Benzoyl-2′-O-(TIPS)-3′-succinoyl-5′-(4,4′-dimethoxytrityl)cytidine (22), wherein TIPS represents the triisopropylsilyl group. See Scheme 5.

N⁴-Benzoyl-2′-O-(TIPS)-5′-(4,4′-dimethoxytrityl)cytidine (21): To a solution containing 10 g (15.4 mmol) of N⁴-Benzoyl-5′-(4,4′-dimethoxytrityl)cytidine and 2.83 mL (41.6 mmol) of imidazole in 40 mL of DMF, 6.6 mL (30.8 mmol) of triisopropylsilyl chloride was added dropwise. After stirring 36 hrs, 100 mL of aq. 5% NaCO₃ was added and the aqueous layer was extracted with two 100-mL portions of diethyl ether. The organic layers were combined, dried over Na₂SO₄ and the solvent was removed under reduced pressure. The crude product was applied to a silica gel column; elution with a gradient of 3:7-4:1 ethyl acetate-petroleum ether provided of N⁴-Benzoyl-2′-O-(TIPS)-5′-(4,4′-dimethoxytrityl)cytidine (1) as a white foam: yield 5.2 g (42%); silica gel TLC: R_{f} 0.55 (4:1 ethyl acetate-petroleum ether). ¹H NMR (CDCl₃) δ 8.53 (bs, 2H), 7.87 (bs, 2H), 7.61 (t, J=7 Hz, 1H), 7.51 (t, J=8 Hz, 2H), 7.42-7.44 (m, 2H), 7.25-7.36 (m, 8H), 6.86-6.89 (m, 4H), 6.04 (d, J=2 Hz, 1H), 4.48-4.50 (m, 1H), 4.40-4.46 (m, 1H), 4.13-4.16 (m, 1H), 3.82 (d, J=1 Hz, 6H), 3.52-3.67 (m, 2H), 2.54 (d, J=8 Hz, 1H), 1.29-1.37 (m, 3H), 1.11 (d, J=7 Hz, 18H).

N⁴-Benzoyl-2′-O-(TIPS)-3′-succinoyl-5′-(4,4′-dimethoxytrityl)cytidine (22):

To a stirred solution containing 500 mg (0.62 mmol) of N⁴-Benzoyl-2′-O-(TIPS)-5′-(4,4′-dimethoxytrityl)cytidine (21) and 98 mg (0.81 mmol) of DMAP in 3 mL of CH₂Cl₂, 81 mg (0.81 mmol) of succinic anhydride was added. After stirring for 12 hrs, 30 mL of CH₂Cl₂ was added and washed with three 20-mL portion of 0.5 M aqueous K₂HPO₄. The organic layer was dried over Na₂SO₄ and the solvent was removed under reduced pressure, providing N⁴-Benzoyl-2′-O-(TIPS)-3′-succinoyl-5′-(4,4′-dimethoxytrityl)cytidine (22) as a white powder: yield 400 mg (71%); silica gel TLC: R_{f} 0.17 (4:1 ethyl acetate-petroleum ether). ¹H NMR (CDCl₃) δ 8.54 (d, J=7 Hz, 1H), 7.93 (d, J=7 Hz, 2H), 7.57 (t, J=7 Hz, 1H), 7.48 (t, J=8 Hz, 2H), 7.38-7.40 (m, 2H), 7.24-7.34 (m, 8H), 6.86 (dd, J=9, 1 Hz, 4H), 6.06 (d, J=3 Hz, 1H), 5.35-5.37 (m, 1H), 4.66 (dd, J=4, 3 Hz, 1H), 4.34 (d, J=7 Hz, 1H), 3.81 (s, 6H), 3.60 (dd, J=11, 2 Hz, 1H), 3.39 (dd, J=11, 2 Hz, 1H), 3.00 (s, 1H), 2.56-2.73 (m, 4H), 1.14-1.24 (m, 3H), 1.05 (d, J=7 Hz, 18H).

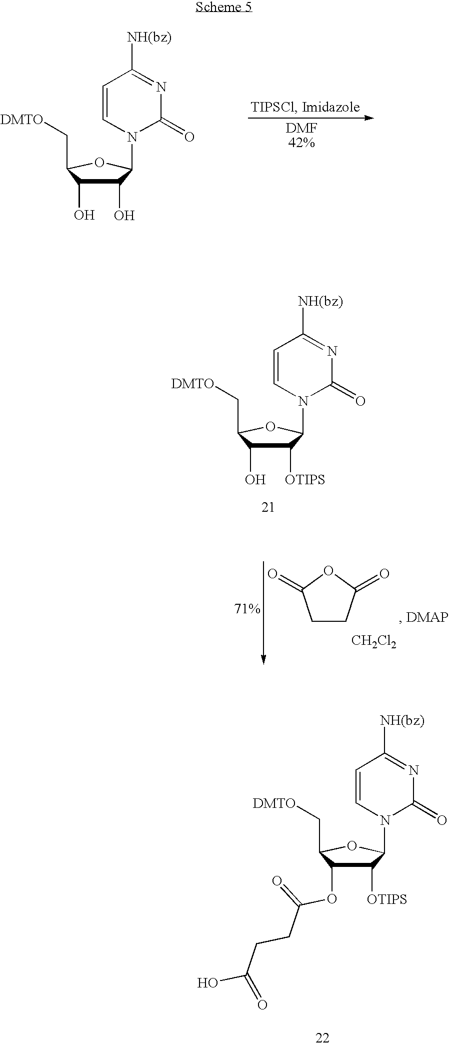

### EXAMPLE 6

This example demonstrates the synthesis of 2′-TIPS-rC CPG support (Formula 4) which was used subsequently for the synthesis of modified oligonucleotides.

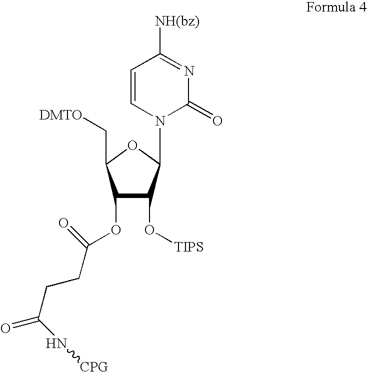

Synthesis of 2′-TIPS-rC CPG support: To a slurry of amino-lcaa-CPG (1.5 g) in 8 mL of acetonitrile were added 0.4 ml of pyridine, 3′-succinyl-2′-TIPS-rC nucleoside (21) (0.14 g, 145 μmoles), DIC (0.157 g, 1 mmole), N-hydroxysuccinimide (6 mg, 50 μmoles) and the reaction mixture was placed on rotary shaker. After 12 hrs the resulting CPG was filtered and washed with CH₃CN (5×50 mL). The CPG was then treated with Ac₂O:Melm:Py (10:10:80) (3×30 mL; 5 minutes each treatment). The derivatized CPG was washed with CH₃CN (5×30 mL), CH₂Cl₂ (3×30 mL), and dried in vacuum overnight. DMT-loading was usually above 25-30 μmol/g.

The analogous support (Formula 5) in which the 2′-modification is tert-butyldiphenylsilyl (TBDPS) was synthesized using the same methods as just described.

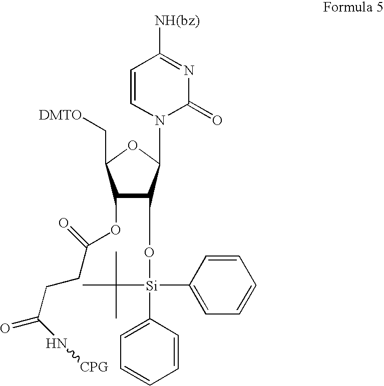

### EXAMPLE 7

The following example demonstrates the ability of 2′-modified nucleosides to block primer extension and/or PCR and function in dual-labeled probe (DLP) applications.

Oligonucleotide Synthesis: Oligonucleotides were synthesized using standard phosphoramidite chemistry (McBride and Caruthers (1983) Tetrahedron Lett., 24:245-248) and purified by HPLC. SEQ ID NOs: 5-10 and 16 (see Table 1) were synthesized on the solid supports described in Examples 3 and 6. In SEQ ID NOs: 9-12, “X” represents dU-aoIBFQ: 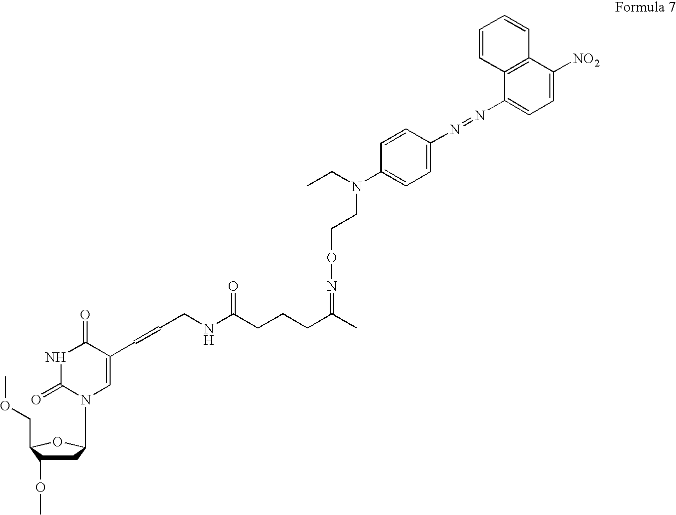
and was introduced with the ketone dU phosphoramidite described by Dey and Shepperd (Org. Lett. (2001) v.3, pp. 3983-3986) followed immediately by conjugation with the aminooxy quencher reagent (3) while the growing oligonucleotide chain was attached to the CPG support. The fluorescein reporter group (FAM) was attached to the 5′-end of SEQ ID NO: 16 using 6-carboxyfluorescein phosphoramidite from Glen Research, Sterling, Va.

Electrospray-ionization liquid chromatography mass spectroscopy (ESI-LCMS) of each oligonucleotide was performed using an Oligo HTCS system (Novatia, Princeton, N.J.), which consisted of ThermoFinnigan TSQ7000, Xcalibur data system, ProMass data processing software and Paradigm MS4™ HPLC (Michrom BioResources, Auburn, Calif.). Protocols recommended by manufacturers were followed. Experimental molar masses for all compounds were within 0.02% of expected molar mass, confirming the identity of the compounds synthesized.

Test system: The Human Enolase gene (Henol, NM_{—}001428) was used as the test system. SEQ ID NO 1 shows a map of the portion of the gene employed in PCR assays. Locations of primers and probes are indicated in underlined bold text. The Henol amplicon is 162 bases using the For1/Rev primer pairs and 120 bases using the For2(probe)/Rev primer pairs.

SEQ. ID NO 1: Human Enolase
GGCTGGCAACTCTGAAGTCATCCTGCCAGTCCCGGCGTTCAATGTCATCA
ATGGCGGTTCTCATGCTGGCAACAAGCTGGCCATGCAGGAGTTCATGATC
CTCCCAGTCGGTGCAGCAAACTTCAGGGAAGCCATGCGCATTGGAGCAGA
GGTTTACCACAACCTGAAGAATGTCATCAAGGAGAAATATGGGAAAGATG
CCACCAATGTGGGGGATGAAGGCGGGTTTGCTCCCAACATCCTGGAGAAT
AAAGAAGGCCTGGAGCTGCTGAAGACTGCTATTGGGAAAGC
SEQ ID NO 2: Henol For1
AACTCTGAAGTCATCCTGCCAGTC
SEQ ID NO 3: Henol For2
ATGGCGGTTCTCATGCTGGCAAC
SEQ ID NO 4: Henol Rev
CTTCAGGTTGTGGTAAACCTCTGC

The following oligonucleotides were synthesized and tested for function as PCR primers. Variants included different 2′-blocking groups, as well as sequences having a perfect match to the target vs. a mismatch “T” base at the position immediately adjacent to the 3′-end (to mimic a dU-aoIBFQ insertion).

TABLE 1
Henol Primer and Probe variants

| | | |
|---|---|---|
| Seq ID No | Name | Sequence |

| | | |
|---|---|---|
| SEQ ID NO 2 | For1 | AAC TCT GAA GTC ATC CTG |
| | | CCA GTC |
| SEQ ID NO 4 | Rev | CTT CAG GTT GTG GTA AAC |
| | | CTC TGC |
| SEQ ID NO 3 | For2 | ATG GCG GTT CTC ATG CTG |
| | | GCA AC |
| SEQ ID NO 5 | For2-TIPS | ATG GCG GTT CTC ATG CTG |
| | | GCA AC-TIPS |
| SEQ ID NO 6 | For2-TBDPS | ATG GCG GTT CTC ATG CTG |
| | | GCA AC-TBDPS |
| SEQ ID NO 7 | For2MM- | ATG GCG GTT CTC ATG CTG |
| | TIPS | GCA TC-TIPS |
| SEQ ID NO 8 | For2MM- | ATG GCG GTT CTC ATG CTG |
| | TBDPS | GCA TC-TBDPS |
| SEQ ID NO 9 | For2Q-TIPS | ATG GCG GTT CTC ATG CTG |
| | | GCA XC-TIPS |
| SEQ ID NO 10 | For2Q- | ATG GCG GTT CTC ATG CTG |
| | TBDPS | GCA XC-TBDPS |
| SEQ ID NO 11 | For2QmC | ATG GCG GTT CTC ATG CTG |
| | | GCA XmC |
| SEQ ID NO 12 | For2Q | ATG GCG GTT CTC ATG CTG |
| | | GCA XC |
| SEQ ID NO 13 | For2MM | ATG GCG GTT CTC ATG CTG |
| | | GCA TC |
| SEQ ID NO 14 | For2mC | ATG GCG GTT CTC ATG CTG |
| | | GCA AmC |
| SEQ ID NO 15 | For2MMmC | ATG GCG GTT CTC ATG CTG |
| | | GCA TmC |
| SEQ ID NO 16 | For2-probe | FAM-ATGGCGGTTCTCATGCTGGCA |
| | | A-IBAOrC |

TIPS & TBDPS = 2′-modifications triisopropylsilyl and tert-butyldiphenylsilyl
“mC” = 2′-O methyl C
“X” = dU-aoIBFQ (Formula 7)
“T” = mismatch base, adjacent to 3′-end
IBAOrC = deprotected Compound 10 cleaved from the CPG support

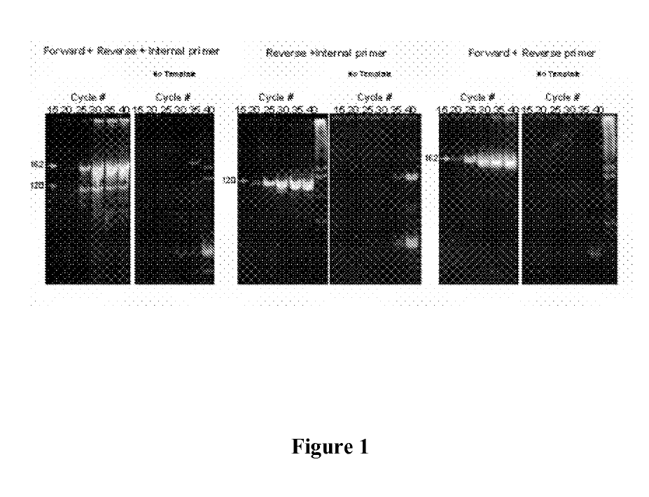

The Henol PCR assay was done using 0.75 units of 1 mmolase DNA Polymerase (Bioline), 3 mM MgCl₂, 800 mM dNTPs, and 200 nM primers using the following cycling program: 95° C. for 10 minutes, then cycle at 95° C. for 15 seconds followed by 60° C. for 1 minute for 15, 20, 25, 30, 35 and 40 cycles. Reaction products were visualized using non-denaturing polyacrylamide gel electrophoresis (PAGE). Cycle numbers were varied to provide semi-quantitative data for relative primer efficiency. Control reactions used unmodified For1 primers, For2 primers or a mixture of For1 and For2 with the Rev primer. The results are shown in FIG. 1. Use of the unmodified For1/Rev primers (Seq ID Nos 2 and 4) gives a strong, single band of the expected size, 162 base pairs. Similarly, use of the unmodified For2/Rev primers (Seq ID Nos 3 and 4) gives a strong, single band of 120 base pairs. Use of mixed unmodified forward primers For1+For2/Rev (Seq ID Nos 2, 3 and 4) resulted in accumulation of significantly more product from the larger For1/Rev amplicon than the smaller For2/Rev amplicon. It is likely that the larger amplicon dominates the reaction not because it is more efficient but rather because the smaller amplicon is degraded by 5′-nuclease action during the process of ongoing simultaneous PCR reactions.

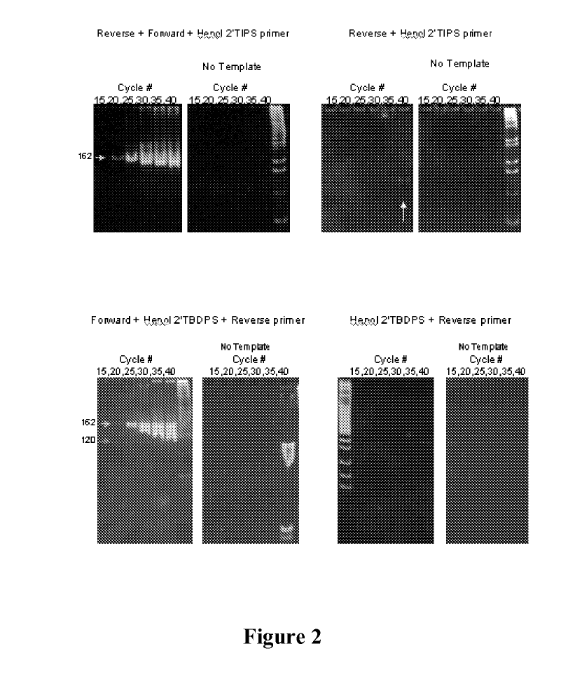

FIG. 2 shows the results obtained using the For2 primer modified on the terminal base with 2′-TIPS and 2′-TBDPS. The 2′-TIPS and 2′-TBDPS modified primers (Seq ID Nos 5 and 6, respectively) do not result in any visible 120 bp band when used with the Rev primer (Seq ID No 4) alone or with competing For1 primer (Seq ID No 2), showing that both of these 2′-modifying group effectively block primer function in PCR. The amplification of the For1 primer in the co-mixture (left panel) rules out the possibility that the modified oligonucleotides contain a general inhibitory factor that interferes with PCR.

Traditionally, 3′-modifications have been employed to block the ability of oligonucleotides to function as primers in PCR. FIG. 2 demonstrates that 2′-modification can also block priming function, even though DNA synthesis occurs via a 3′-internucleoside phosphate linkage formation and 2′-blocked primers have an unblocked 3′-hydroxyl group present that could theoretically support priming and DNA synthesis.

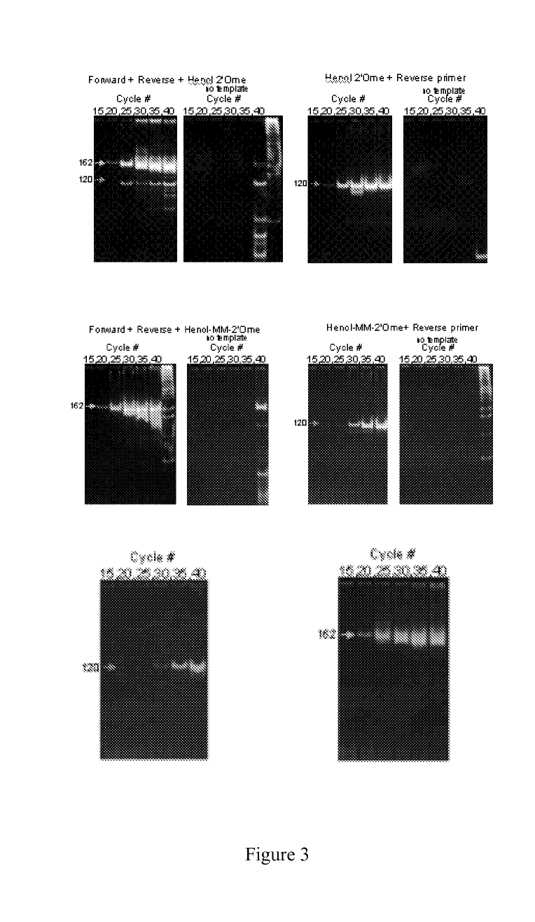

The remaining oligonucleotides shown in Table 1 were similarly tested for their ability to support PCR alone or in the presence of a competing reaction from the For1 primer. FIG. 3 illustrates that not all 2′-modifying groups block priming and PCR. Placement of a 2′OMe group, which is much smaller and less bulky than the TIPS or TBDPS groups, does not severely impair priming function. The For2mC primer (Seq ID No 14) functions nearly as well as the unmodified For2 primer (Seq ID No 3) and does produce a 120 bp amplicon in the presence of a competing reaction from the For1 (Seq ID No 2) primer (see top panel in FIG. 3). Primer For2mM (Seq ID No 15) has the same terminal 2′OMe-C base with an unblocked 3′-end and also has a mismatch (A→T) base change at the adjacent position (−1 from the 3′-end). This primer is less efficient in supporting PCR than the For2mC primer and the expected 120 bp band does not appear until 30 cycles of PCR (see middle panel in FIG. 3). Further, this reaction is not robust and does not survive to produce a visible product in the face of a competing PCR reaction with the For1 primer. Primer For2QmC (Seq ID No 11) adds a bulky hydrophobic modification to the mismatch base at position −1 as a dU-aoIBFQ modification. This primer is even less efficient in supporting priming and PCR than the other 2′-OMe-C primers tested (see bottom panel in FIG. 3). Nevertheless, all of these primers do support priming and PCR to some degree whereas the 2′-TIPS and 2′-TBDPS primers did not support priming and PCR at all.

The relative performance of each of the For2 variant primers described in Table 1 in supporting priming and PCR when paired with the Rev primer (Seq ID No 4) is summarized in Table 2. “Ct” indicates the PCR cycle number where the expected 120 bp band was detectable by visual assay (cycles 15, 20, 25, 30, 35, and 40 were tested). “Comp”+ or − indicates whether the expected 120 bp band was visualized when a competing PCR reaction was included using the For1 (Seq ID No 2) primer. Oligonucleotides with the 2′-TIPS or 2′-TBDPS modifications at their 3′-end were effectively blocked as primers for PCR.

TABLE 2
Summary of PCR gel data

| | | | | |
|---|---|---|---|---|
| Name | Seq ID No | Sequence | Ct | Comp |

| | | | | |
|---|---|---|---|---|
| For2 | Seq ID No 3 | ATGGCGGTTCTCATGC | 20 | + |
| | | TGGCAAC | | |
| For2MM | Seq ID No 13 | ATGGCGGTTCTCATGC | 20 | + |
| | | TGGCATC | | |
| For2Q | Seq ID No 12 | ATGGCGGTTCTCATGC | 20 | + |
| | | TGGCAXC | | |
| For2mC | Seq ID No 14 | ATGGCGGTTCTCATGC | 25 | + |
| | | TGGCAAmC | | |
| For2MMmC | Seq ID No 15 | ATGGCGGTTCTCATGC | 30 | − |
| | | TGGCATmC | | |
| For2QmC | Seq ID No 11 | ATGGCGGTTCTCATGC | 35 | − |
| | | TGGCAXmC | | |
| For2-TIPS | Seq ID No 5 | ATGGCGGTTCTCATGC | >40 | − |
| | | TGGCAAC-TIPS | | |
| For2MM-TIPS | Seq ID No 7 | ATGGCGGTTCTCATGC | >40 | − |
| | | TGGCATC-TIPS | | |
| For2Q-TIPS | Seq ID No 9 | ATGGCGGTTCTCATGC | >40 | − |
| | | TGGCAXC-TIPS | | |
| For2-TBDPS | Seq ID No 6 | ATGGCGGTTCTCATGC | >40 | − |
| | | TGGCAAC-TBDPS | | |
| For2MM- | Seq ID No 8 | ATGGCGGTTCTCATGC | 40 | − |
| TBDPS | | TGGCATC-TBDPS | | |
| For2Q- | Seq ID No 10 | ATGGCGGTTCTCATGC | 40 | − |
| TBDPS | | TGGCAXC-TBDPS | | |

“T”, “X”, TIPS and TBDPS are the same as in Table I
Ct = cycle number threshold for detection of the 120 base pair amplicon
Comp = indicates whether a 120 bp product was detected when competing unmodified For-1 primer (SEQ ID NO: 2) was also employed

### EXAMPLE 8

The following example compares the functional performance of a fluorescent dual-labeled probe (DLP) employing a Fam reporter and an internal quencher with an unblocked 3′-OH but with a 2′-TIPS group (SEQ ID NO: 17) with a traditional 3′-blocked probe containing an aminooxy conjugated IBFQ quencher (SEQ ID NO: 18). In SEQ ID NO: 18, the quencher IBFQ is attached to the 3′-OH as shown in Formula 8.

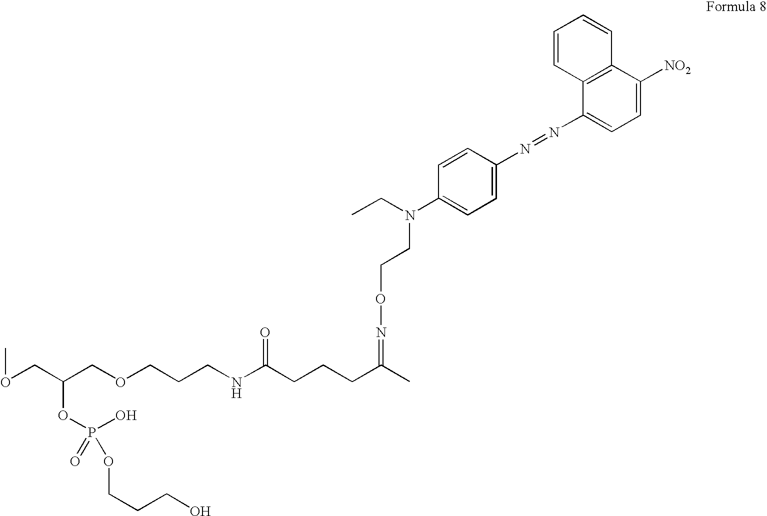

TABLE 3

| | | |
|---|---|---|
| Name | Seq ID No. | Sequence |

| | | |
|---|---|---|
| For2 IB(t9) | Seq ID No 17 | Fam-ATGGCGGTXCTCATGCTGGCAA |
| 2′TIPS | | C-TIPS |
| For2 3′IBFQ | Seq ID No 18 | Fam-AATGGCGGTTCTCATGCTGGCA |
| | | ACAA-IBFQ |

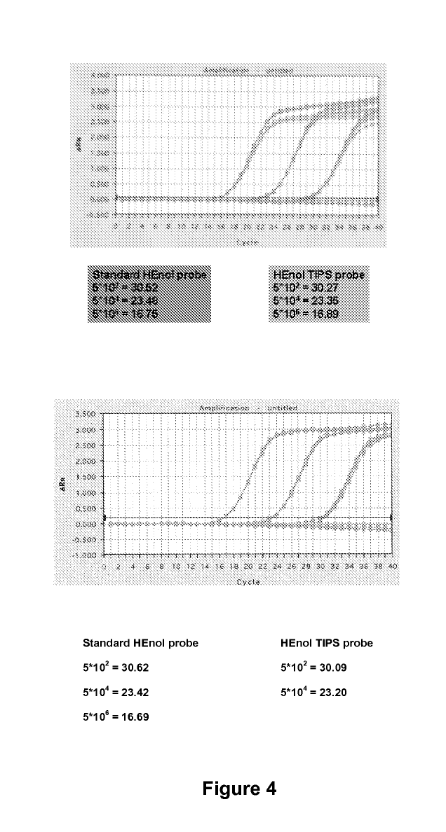

Quantitative real-time PCR (qPCR, or “Taqman”) assays employing the human enolase amplicon (Seq ID No 1) were carried out using 0.75 units of 1 mmolase DNA Polymerase (Bioline), 3 mM MgCl₂, 800 mM dNTPs, 200 nM primers, 200 nM probe with the following cycling parameters: 95° C. for 10 minutes, then cycle at 95° C. for 15 seconds followed by 60° C. for 1 minute for 40 cycles. Input target amounts were 5×10², 5×10⁴, and 5×10⁶ copies of cloned plasmid DNA. All data points were performed in triplicate. Results are shown in FIG. 4. Testing was performed twice with the result that Ct values were identical between probes at all target levels tested. No functional difference was seen between the two probes. Curves generated using both probes are superimposed and are indistinguishable. Therefore 2′-blocking groups of the invention can be used not only to block primer function but also can be incorporated into fluorescence quenched probes useful in Taqman® and other hybridization assays.

### EXAMPLE 9

The following example demonstrates the synthesis of a rU analog with a ketone functionality at the 2′ position attached to a CPG support. See Scheme 6.

5′-O-DMT-2′-O-penten-4-yl-uridine (24) 5′-O-DMT-uridine (1.09 g, 2 mmol) and dibutyltin oxide (0.5 g, 2 mmol) were dried in a vacuum over P₂O₅ for 2 hours, and then were suspended in anhydrous benzene (50 mL) under Ar atmosphere. After refluxing the suspension for 2 hours a clear solution was formed. It was then cooled down to room temperature and the solvent was removed in a vacuum giving stannylene derivative 23 as a white crystalline solid. The stannylene derivative was used in the next step without further purification. It was dissolved in anhydrous DMF (10 mL) and treated with 1-iodo-4-pentene (3 mL, 15 mmol) under Ar at 80° C. for 36 hours. The solvent was evaporated in vacuum and the residue was partitioned between EtOAc and saturated NaHCO₃. The organic layer was separated and washed with saturated NaHCO₃, then brine, and then dried over Na₂SO₄. The product (R_{f}˜0.5 in EtOAc:Hexane (2:1)) was isolated by flash chromatography on silica gel separating from 3′-O-isomer (R_{f}˜0.35). The same mixture of solvents was used to elute the product. Fractions containing pure product were pooled and concentrated in vacuum to give 410 mg (33% from theory) of yellowish foam. The product was 95% pure by HPLC. ¹H NMR (DMSO-d₆) δ 1.61-1.68 (m, 2H), 2.12 (q, 2H), 3.25-3.38 (m, 2H), 3.56-3.69 (m, 2H), 3.78 (s, 6H), 3.94 (t, 1H), 4.00-4.03 (m, 1H), 4.22 (q, 1H), 4.96-5.06 (m, 2H), 5.18 (d, 1H), 5.33 (dd, 1H), 5.79-5.90 (m, 1H), 5.85 (d, 1H), 6.94 (d, 4H), 7.25-7.44 (m, 9H), 7.77 (d, 1H), 11.42 (br.d, 1H).

5′-O-DMT-2′-O-(4-oxopentyl)uridine (25) Compound 24 (250 mg, 0.41 mmol) was dissolved in DMF (10 mL) and 1 mL of water was added. To the resulting solution Pd Cl₂ (35 mg, 0.20 mmol) and Cu(OAc)₂ monohydrate (100 mg, 0.50 mmol) were added with stirring. The air in the flask was replaced with oxygen and the reaction suspension was vigorously stirred at room temperature under oxygen atmosphere. The reaction was monitored by HPLC. The starting material almost disappeared after 18 hours. The reaction was allowed to proceed for another 24 hours, the solvent was evaporated, and the residue was partitioned between EtOAc and brine. The organic layer washed with brine, dried over Na₂SO₄ and concentrated to a syrup. TLC in EtOAc revealed one major product with R_{f}˜0.36. The product was isolated by flash chromatography on silica gel loading with EtOAc:Hexane (2:1) and eluting with pure EtOAc. The yield was 113 mg (43% theory). The structure was confirmed by ¹H NMR (DMSO-d₆) δ 1.89-1.97 (m, 2H), 1.95 (s, 3H), 2.50 (t, 2H), 3.66 (s, 6H), 3.61-4.06 (m, 6H), 4.28-4.31 (m, 1H), 4.57-4.62 (m, 1H), 4.86-4.91 (m, 1H), 5.67 (d, 1H), 6.93-6.99 (m, 4H), 7.26 (t, 1H), 7.37 (t, 2H), 7.56 (d, 4H), 7.72 (d, 2H), 8.24 (d, 1H), 13.40 (br.s, 1H).

5′-O-DMT-2′-O-(4-oxopentyl)uridine-3′-O-succinate, PFP-ester (26) Compound 25 (113 mg, 0.18 mmol) was dissolved in anhydrous pyridine (5 mL). dimethylamine puridine (DMAP) (24 mg, 0.2 mmol) and succinic anhydride (50 mg, 0.5 mmol) were added, and the reaction mixture was stirred until everything went into the solution. The reaction mixture was kept at room temperature and monitored by HPLC. After 36 hours almost all the starting material had disappeared. Water (0.5 mL) was added and the reaction mixture was left at room temperature overnight to hydrolyze the excess anhydride. The solvents were evaporated and the residue was partitioned between EtOAc and 10% aqueous citric acid. The organic layer was separated and washed with water, saturated NaHCO₃, and then brine. It was then dried over MgSO₄, concentrated to dryness and used for PFP-ester preparation without further purification.

The 3′-O-succinate derivative was dried in vacuum overnight and dissolved in DCM (5 mL) containing DIEA (174 μL). Pentafluorophenyl trifluoroacetate (86 mL, 0.5 mmol) was added and the reaction was allowed to proceed at room temperature for 30 minutes. TLC analysis in EtOAc revealed one major spot R_{f}˜0.60. The reaction mixture was diluted with EtOAc:Hexane (1:1) and loaded on silica gel column. The column was washed with 2 bed volumes of the same solvent system, and the product was eluted with pure EtOAc. Fractions containing the pure product (26) were pooled and concentrated in vacuum affording 107 mg of TLC pure material as a yellowish glass. ¹H NMR was recorded to confirm the structure: (CD₃CN) δ 1.68-1.75 (m, 2H), 1.95-1.98 (m, 2H), 2.06 (s, 3H), 2.44 (t, 2H), 2.82-2.86 (m, 2H), 3.03-3.07 (m, 2H), 3.40-3.60 (m, 4H), 3.79 (s, 6H), 4.19-4.22 (m, 2H), 5.31 (t, 1H), 5.40 (dd, 1H), 5.89 (d, 1H), 6.87-6.92 (m, 4H), 7.25-7.36 (m, 7H), 7.42-7.46 (m, 2H), 7.64 (d, 1H), 9.17 (s, 1H).

Attachment of 26 to CPG Two grams of LCAA CPG (Prime Synthesis; 1000 Å; 79 μmol/g) was treated with PFP-ester 26 (107 mg; 119 μmol) in CH₃CN (8 mL) containing DIEA (400 μL) overnight at room temperature with gentle shaking. The modified CPG (27) washed with CH₃CN and capped by treatment with acetic anhydride. Finally, the CPG 27 washed with CH₃CN, CH₂Cl₂ and dried in vacuum. DMT loading was measured to be 37.1 μmol/g.

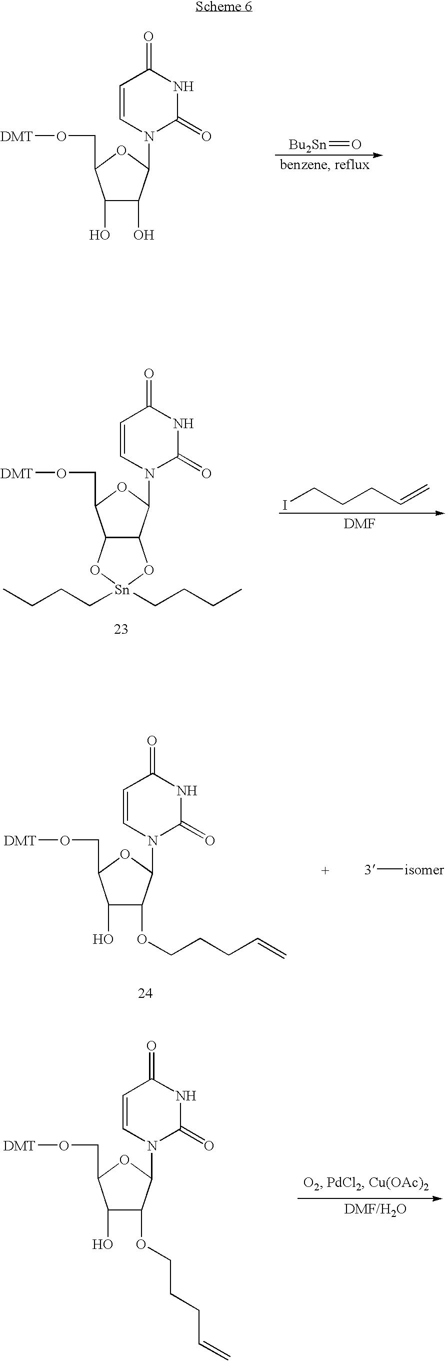

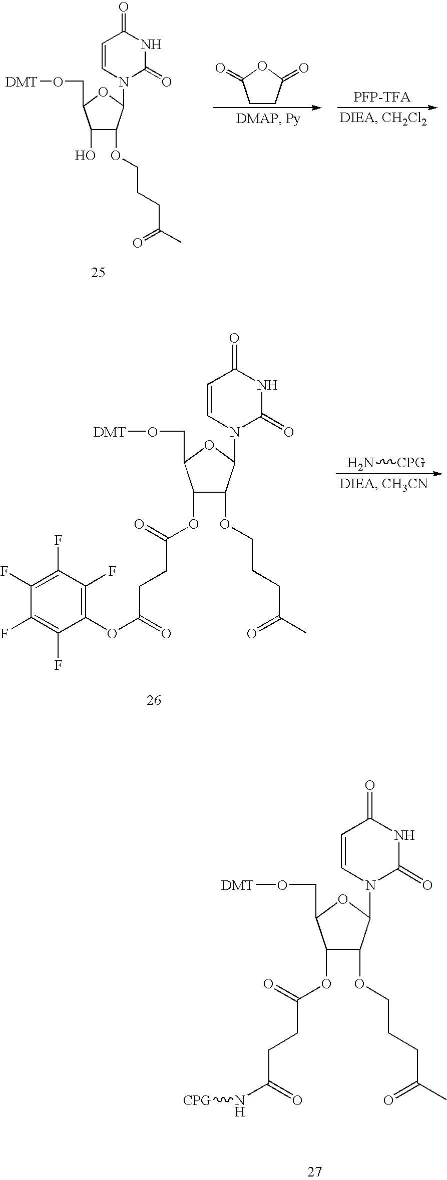

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A chemical composition having a structure of Formula 9: 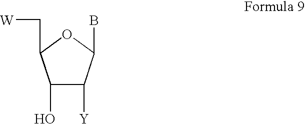
wherein B is a nucleobase;
Y is a fluorescent dye, a quenching dye or an acetal group;
and W is selected from a group comprising a phosphodiester bond, a hydroxyl group, a protected hydroxyl group, a nucleotide, an oligonucleotide chain, an —SH— group, a protected —SH— group or a phosphorothioate bond.

2. An oligonucleotide between about 10 and about 75 monomers in length wherein at least one of said monomers is the composition of claim 1.

3. The oligonucleotide of claim 2 wherein the claim 1 composition is at the 3′-end of the oligonucleotide.

4. The oligonucleotide of claim 2 wherein the oligonucleotide is complementary to a target nucleic acid sequence for which the oligonucleotide is a probe.

5. The oligonucleotide of claim 4 wherein the oligonucleotide is modified with a reporter group.

6. The oligonucleotide of claim 5 wherein the reporter group is a fluorophore.

7. The oligonucleotide of claim 5 wherein the oligonucleotide also contains a quencher.

8. A method of detecting a target nucleic acid sequence wherein the oligonucleotide of claim 4 is used as a probe.

9. The method of claim 8 wherein the probe is used to detect a product of an amplification reaction.

10. The method of claim 9 wherein said amplification reaction is PCR or a polynomial amplification reaction.

11. The method of claim 10 wherein the amplification reaction utilizes an enzymatic cleavage of the oligonucleotide.

12. The method of claim 11 wherein the enzymatic cleavage of the oligonucleotide is effected by a 5′-nuclease activity of a DNA polymerase.

13. A method of detecting a polynomial amplification product wherein an oligonucleotide of claims 2 is used as a template for polymerase extension of said polynomial amplification product.

14. The method of claim 13 wherein said oligonucleotide is cleaved enzymatically.

15. An oligonucleotide between about 10 to about 75 monomers in length, wherein at least one monomer has a chemical composition having a structure of Formula 10: 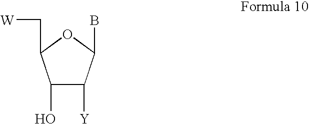
wherein B is a nucleobase;
Y is a fluorescent dye, a quenching dye, a silyl group, a ketone group or an acetal group;
and W is selected from a group comprising a phosphodiester bond, a hydroxyl group, a protected hydroxyl group, a nucleotide, an oligonucleotide chain, an —SH— group, a protected —SH— group or a phosphorothioate bond.

16. The oligonucleotide of claim 15 wherein a monomer having a chemical composition having a structure of Formula 10 is incorporated at the 3′-end of the oligonucleotide.

17. The oligonucleotide of claim 15 wherein the oligonucleotide is complementary to a target nucleic acid sequence for which the oligonucleotide is a probe.

18. The oligonucleotide of claim 17 wherein the oligonucleotide is modified with a reporter group.

19. The oligonucleotide of claim 18 wherein the reporter group is a fluorophore.

20. The oligonucleotide of claim 18 wherein the oligonucleotide also contains a quencher.

21. The oligonucleotide of claim 16 wherein Y of the 3′-end monomer inhibits extension of the oligonucleotide by a polymerase.

22. The oligonucleotide of claims 15 wherein Y is a triisopropyl silyl or a tert-butyldiphenylsilyl group.

23. A method of detecting a target nucleic acid sequence wherein the oligonucleotide of claim 16 is used as a probe.

24. The method of claim 23 wherein the probe is used to detect a product of an amplification reaction.

25. The method of claim 24 wherein said amplification reaction is PCR or a polynomial amplification reaction.

26. The method of claim 25 wherein the amplification reaction utilizes an enzymatic cleavage of the oligonucleotide.

27. The method of claim 26 wherein the enzymatic cleavage of the oligonucleotide is effected by a 5′-nuclease activity of a DNA polymerase.

28. A method of detecting a polynomial amplification product wherein an oligonucleotide of claims 15 is used as a template for polymerase extension of said polynomial amplification product.

29. The method of claim 28 wherein said oligonucleotide is cleaved enzymatically.

30. A chemical composition having the structure of Formula 11: 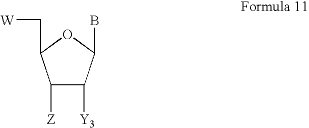
wherein B is a nucleobase,
Y₃ is XR wherein X is a heteroatom or an alkyl group and R is a substituted alkyl or acetal chain with a ketone;
W is selected from a group comprising a phosphodiester bond, a hydroxyl group, a protected hydroxyl group, a nucleotide, an oligonucleotide chain, an —SH— group, a protected —SH— group or a phosphorothioate bond;
And Z is selected from a group comprising a hydroxyl group, one or more nucleotides, a solid support or a linking group.

31. The chemical composition of claim 30 wherein the linking group is a phosphoramidite, a succinate monoester, H-phosphonate or a phosphate diester.

32. The composition of claim 30 wherein R is (CH₂)ₙ—X₂—(CH₂)ₙ—(X₃)ₘ, wherein X₂ is a heteroatom, a ketone group or CH₂, n is 1-5, X₃ is a COCH₃ group or a ketone group, and m is 0 when X₂ is a ketone or 1 when X₂ is not a ketone group.

33. The composition of claim 30 wherein Y₃ is

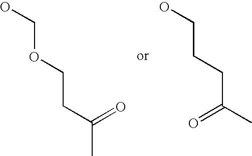

34. The composition in claims 30 wherein the ketone group is conjugated to an aminooxy label.

35. The composition of claim 34 wherein the aminooxy label is a fluorophore.

36. The composition of claim 34 wherein the aminooxy label is a quencher.

37. The composition of claim 34 wherein the composition is Formula 12:

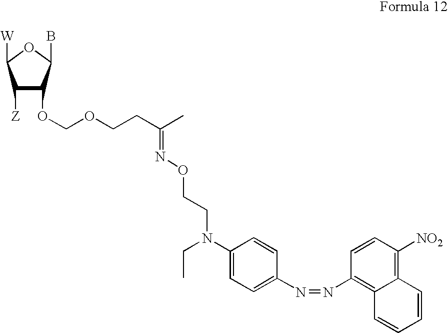

38. An oligonucleotide between about 10 and about 75 monomers in length wherein at least one of said monomers is the composition of claim 30.

39. The oligonucleotide of claim 38 wherein the claim 30 composition is at the 3′-end of the oligonucleotide.

40. The oligonucleotide of claim 38 wherein the oligonucleotide is complementary to a target nucleic acid sequence for which the oligonucleotide is a probe.

41. The oligonucleotide of claim 40 wherein the oligonucleotide is modified with a reporter group.

42. The oligonucleotide of claim 41 wherein the reporter group is a fluorophore.

43. The oligonucleotide of claim 42 wherein the oligonucleotide also contains a quencher.

44. A method of detecting a target nucleic acid sequence wherein the oligonucleotide of claim 41 is used as a probe.

45. The method of claim 44 wherein the probe is used to detect a product of an amplification reaction.

46. The method of claim 45 wherein said amplification reaction is PCR or a polynomial amplification reaction.

47. The method of claim 46 wherein the amplification reaction utilizes an enzymatic cleavage of the oligonucleotide.

48. The method of claim 47 wherein the enzymatic cleavage of the oligonucleotide is effected by a 5′-nuclease activity of a DNA polymerase.

49. A method of detecting a polynomial amplification product wherein an oligonucleotide of claims 38 is used as a template for polymerase extension of said polynomial amplification product.

50. The method of claim 49 wherein said oligonucleotide is cleaved enzymatically.

51. An oligonucleotide between about 10 to about 75 monomers for use as a probe in a hybridization assay, said oligonucleotide being complementary to a target nucleic acid sequence, wherein said oligonucleotide contains a reporter group, and wherein a monomer at the 3′-end of the oligonucleotide is Formula 13: 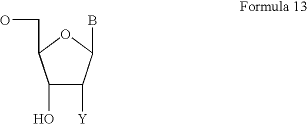
wherein Y is a blocking group that inhibits extension of the oligonucleotide by a polymerase.

52. The oligonucleotide of claim 51 wherein the reporter group is a fluorophore.

53. The oligonucleotide of claim 51 wherein the oligonucleotide also contains a quencher.

54. A method of detecting a target nucleic acid sequence wherein the oligonucleotide of claim 52 is used as a probe.

55. The method of claim 54 wherein the probe is used to detect a product of an amplification reaction.

56. The method of claim 55 wherein said amplification reaction is PCR or a polynomial amplification reaction.

57. The method of claim 56 wherein the amplification reaction utilizes an enzymatic cleavage of the oligonucleotide.

58. The method of claim 57 wherein the enzymatic cleavage of the oligonucleotide is effected by a 5′-nuclease activity of a DNA polymerase.

59. A method of detecting a polynomial amplification product wherein an oligonucleotide of claims 51 is used as a template for polymerase extension of said polynomial amplification product.

60. The method of claim 59 wherein said oligonucleotide is cleaved enzymatically.

61. A kit for an amplification of a target sequence, the kit comprising:
a) the oligonucleotide probe of claim 51;
b) a set of one or more target sequence primers;
c) a polymerase enzyme.

62. A nucleic acid comprising: (a) a cleavage domain comprising a single-stranded region, said single-stranded region comprising at least one internucleotide linkage 3′ to an adenosine residue, at least one internucleotide linkage 3′ to a cytosine residue, at least one internucleotide linkage 3′ to a guanosine residue, and at least one internucleotide linkage 3′ to a uridine residue, and wherein said cleavage domain does not comprise a deoxyribonuclease-cleavable internucleotide linkage; (b) a fluorescence reporter group on one side of the internucleotide linkages; and (c) the monomer of claim 37 on the other side of the internucleotide linkages.
